(19) United States
(12) Patent Application Publication
(10) Pub. No.: US 2010/0075984 A1
(43) Pub. Date: Mar. 25, 2010

(54) DICARBONYLIC COMPOUNDS WITH ANTIBACTERIAL ACTIVITY

(75) Inventors: José Hidalgo Rodríguez, Sant Fost de Campsentelles (ES); Juan Lorenzo Catena Ruiz, Cubelles (ES); Isabel Masip Masip, Barcelona (ES); Maria del Carmen Serra Comas, L'Hospitalet de Llobregat (ES); Oscar Rey Puiggrós, Barcelona (ES); Carmen Lagunas Arnal, Barcelona (ES); Carolina Salcedo Roca, Corbera (ES); Dolors Balsa López, Badalona (ES)

Correspondence Address: ROTHWELL, FIGG, ERNST & MANBECK, P.C., 1425 K STREET, N.W., SUITE 800, WASHINGTON, DC, 20005, US

(73) Assignee: LABORATORIOS SALVAT, S.A., Esplugues de Llobregat (Barcelona) (ES)

(21) Appl. No.: 12/161,391

(22) PCT Filed: Jan. 18, 2007
(86) PCT No.: PCT/EP07/50489

§ 371 Date: Jul. 18, 2008

(30) Foreign Application Priority Data
Jan. 19, 2006 (ES) P 200600158

(51) Int. Cl. A61K 31/497; C07D 413/10

(52) U.S. Cl. 514/254.04; 544/367

(57) ABSTRACT

Compounds of formula (I), and their pharmaceutically acceptable salts and solvates, wherein X represents —O—, —NH—, —S—, —NHC(═O)— or —NHC(═S)—; R1 represents —H or a hydrocarbon chain; R2 represents —H, alkoxy, amino, a hydrocarbon chain or a radical of a cycle; R3 represents —H, a hydrocarbon chain or a radical of a cycle; R4 represents —H or a hydrocarbon chain; alternatively R3 and R4 form together a cycle; R5 and R6 represent —H or halogen, and R7 represents —H, a hydrocarbon chain or heteroaryl, are useful against bacterial infections in animals, including humans.

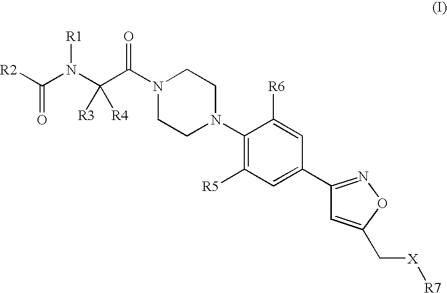

The present invention relates to dicarbonylic compounds with antibacterial activity, as well as to pharmaceutical compositions containing them and to their use in medicine.

### BACKGROUND OF THE ART

The international microbiological community continues to express serious concern in view of the alarming increase of resistance to commercially available antibiotics, which reduces the range of possibilities of treatment of the different infectious processes. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens, for example staphylococci, enterococci, and streptococci, are particularly important due to the development of the resistant strains which are both difficult to treat and eradicate from the hospital environment. Examples of such strains are methicillin resistant staphylococci, methicillin resistant coagulase negative staphylococci, penicillin resistant Streptococcus pneumoniae and several vancomycin resistant enterococci.

Until oxazolidinones came out, the best clinically effective antibiotic for the treatment of such resistant Gram-positive pathogens was vancomycin. Vancomycin is a glycopeptide that shows certain nephrotoxicity an ototoxicity as well as low bioavailability and as a consequence it is parenterally administered. Nevertheless, antibacterial resistance to vancomycin and other glycopeptides is also appearing and this resistance is increasing, rendering these agents less and less effective in the treatment of infections produced by Gram-positive pathogens.

From 1989, diverse antibacterial compounds containing an oxazolidinone ring have been described, in particular eperezolid and linezolid both of Pharmacia Corporation (S. J. Brickner et al., J. Med. Chem. 1996, 39, 673-679). From them, only linezolid is commercially available at present.

Though the discovery of the mentioned oxazolidinones means a clear advance in the treatment of infections produced by Gram-positive pathogens, it is worth noting that bacterial resistance to known antibacterial agents may be developed, for example, by mutation of active binding sites in the bacteria rendering a decrease or total loss of activity of the previously active pharmacophore. Therefore, it is useful to obtain new antibacterial agents without crossed resistances.

WO 03/008395 A1 describes the preparation of antibacterial compounds structurally related to the compounds of the invention. Those compounds are emcompassed by the following general formula

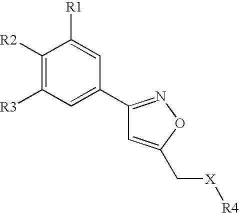

wherein, among other meanings, R2 may represent:

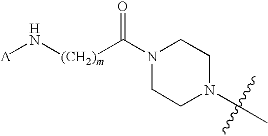

wherein A represents —H, (C₁-C₃)alkyl, vinyl, allyl, ethynyl, propargyl, phenyl or a radical of an optionally substituted aromatic ring system and m represents a value from 0 to 8.

The background art illustrates the present interest in providing new compounds with antibacterial activity preferably with broad spectrum of activity, particularly against staphylococci or enterococci resistant to other antibiotics, the main cause of multiresistant hospital infections.

### SUMMARY OF THE INVENTION

The present invention relates to dicarbonylic compounds of general formula I,

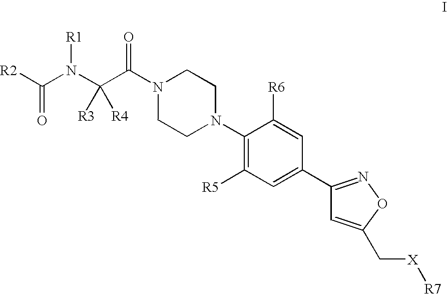

their stereoisomers and mixtures thereof, its polymorphs and mixtures thereof, N-oxides, when there are oxidable nitrogen atoms, and the pharmaceutically acceptable solvates and addition salts thereof, wherein:
X represents —O—, —NH—, —S—, —NHC(═O)— or —NHC(═S)—;
R1 represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra;
R2 represents —H, —ORb, —NRbRc, —(C₁-C₄)alkyl, —(C₂-C₄)alkenyl, —(C₂-C₄)alkynyl, or -Cy1 optionally substituted with one or more groups Rd or Re, wherein —(C₁-C₄)alkyl, —(C₂-C₄)alkenyl or —(C₂-C₄)alkynyl may be optionally substituted with one or more groups Rd and/or one group Rf;
R3 represents R1 or -Cy2 optionally substituted with one or more groups Ra or Rc;
R4 represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted by one or more halogen atoms;
alternatively, R3 and R4 may form together a 3- to 7-membered monocyclic ring, partially unsaturated, saturated or aromatic, containing from one to three heteroatoms independently selected from O, S and N, optionally substituted at any available position by one or more substituent Rc or halogen atoms;
R5 and R6 independently represent —H or halogen;
R7 represents R4 or 5- or 6-membered heteroaryl, containing from one to three heteroatoms independently selected from O, S and N, optionally substituted with one or more groups Rc or halogen atoms;
each Ra independently represents halogen, ═O, —ORc, —OC(═O)Rc, ═CRcRc, —CN, —C(═O)Rc, —C(═O)ORc, —C(═O)NRcRc, —NO2, —NRcRc, —NRcC(═O)Rc, —NRcC(═O)ORc or —NRcC(═O)NRcRc;
Rb represents —H, Rg, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra and/or one group Rg;
each Rc independently represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted by one or more halogen atoms;
each Rd independently represents halogen, ═CRaRc, ═CRcRc, —CN, —C(═O)Re′, —C(═O)ORe′, —C(═O)NRe′Rh′, —C(═O)SRe′, —C(═NRh′)NRe′Rh′, —C(═NRe′)NRh′Rh′, —C(═S)ORe′, —C(═S)SRe′, —ORe′, ═O, —OC(═O)Re′, —OC(═O)NReRh′, —OC(═S)Re′, —O—N═O, —OSO₂Re, —NRe′Rh′, ═NRe′, ═N—CN, ═N—ORe′, —N⁺Re′Rh′Rh′, —N═NRe′, —NRh′-NRe′Re′, —NRe′-NRe′Rh′, —N₃, —N═O, —NRh′ORe′, —NRe′ORh′, —NO₂, —NRe′C(═O)Rh′, —NRh′C(═O)Re′, —NRh′C(═O)ORe, —NRe′C(═O)ORh, —NRh′C(═O)NReRh′, —NRe′C(═O)NRhRh′, —NRe′C(═O)NRh′NRh′Rh′, —NRh′C(═O)NRe′NRh′Rh′, —NRh′C(═O)NRh′NRe′Rh′, —NRe′SO₂Rh′, —NRh′SO₂Re′, —SRe′, —SORe′, —SO₂Re, —SO₂NRe′Rh′ or —SO₂ORe′;
each Re independently represents Rf or —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra and/or one group Rg;
each Re′ independently represents —H or —Re;
each Rf independently represents -Cy1 optionally substituted with one or more groups Ra or Rh;
each Rg independently represents -Cy1 optionally substituted with one or more groups Ra or Rc;
each Rh independently represents —(C₁-C₄)alkyl, —(C₂-C₄)alkenyl or —(C₂-C₄)alkynyl, all of them optionally substituted with one or more groups Ra;
each Rh′ independently represents —H or —Rh;
Cy1 represents a C- or N-radical of a 3- to 7-membered monocyclic or 6- to 10-membered bicyclic ring system, partially unsaturated, saturated or aromatic, containing from one to three heteroatoms independently selected from O, S and N; and
Cy2 represents a C- or N-radical of a 3- to 7-membered monocyclic ring, partially unsaturated, saturated or aromatic, containing from one to three heteroatoms independently selected from O, S and N.

In the previous definitions, the term (C₁-C₄)alkyl represents a straight or branched saturated hydrocarbon chain containing from one to four carbon atoms, for example methyl, ethyl, propyl, isopropyl, butyl, isobutyl, sec-butyl and tert-butyl. The term (C₂-C₄)alkenyl represents an unsaturated straight or branched saturated hydrocarbon chain containing from two to four carbon atoms and one or more double bonds, for example ethenyl, 1-propenyl, 2-propenyl, 1-butenyl, 2-butenyl, 3-butenyl and 1,3-butadienyl. The term (C₂-C₄)alkynyl represents an unsaturated straight or branched saturated hydrocarbon chain containing from two to four carbon atoms and one or more triple bonds, for example ethinyl, 1-propynyl, 2-propynyl, 1-butynyl, 2-butynyl, 3-butynyl and 1,3-butadinyl. The groups (C₁-C₄)alkyl, (C₂-C₄)alkenyl and (C₂-C₄)alkynyl may be optionally substituted according to the description whenever appropriate from a chemical view point.

The term halogen represents a radical of fluoro, chloro, bromo or iodo. A group ═O may be attached to a carbon atom to form —C(═O)— or to a sulfur atom to form —S(═O)— or —S(═O)₂—.

The term heteroaryl represents a C- or N-radical of an aromatic 5- or 6-membered monocyclic ring, containing from one to four heteroatoms independently selected from O, S and N, that may be substituted according to the description at any available ring position. Examples include, among others, radicals of pyrrol, furan, thiophene, imidazole, isoxazole, isothiazole, oxazole, 1,2,4-oxadiazole, 1,2,4-thiadiazole, 1,3,4-oxadiazole, 1,3,4-thiadiazole, 1,2,3-triazole, 1,2,4-triazole, pyridine, pyrimidine, pyridazine and pyrazine.

The term Cy1 represents a C- or N-radical of a 3- to 7-membered monocyclic or 6- to 10-membered bicyclic ring system, partially unsaturated, saturated or aromatic. The term Cy2 represents a C- or N-radical of a 3- to 7-membered monocyclic ring, partially unsaturated, saturated or aromatic. Both Cy1 and Cy2 may contain from one to four heteroatoms independently selected from O, S and N, and may be substituted according to the description at any available ring position. Examples of Cy1 and Cy2 include, among others, radicals of cyclopropane, cyclobutane, cyclopentane, cyclohexane, cycloheptane, aziridine, dihydrofuran, pyrroline, pyrazoline, oxirane, oxethane, imidazolidine, isothiazolidine, isoxazolidine, oxazolidine, pyrazolidine, pyrrolidine, thiazolidine, dioxane, morpholine, piperazine, piperidine, pyran, tetrahydropiran, azepine, oxazine, oxazoline, pyrroline, thiazoline, pyrazoline, imidazoline, isoxazoline, isothiazoline, phenyl, naphthy, 1,2,4-oxadiazole, 1,2,4-thiadiazole, 1,3,4-oxadiazole, 1,3,4-thiadiazole, furan, imidazole, isoxazole, isothiazole, oxazole, pyrazole, pyrrole, thiazole, thiophene, 1,2,3-triazole, 1,2,4-triazole, pyrazine, pyridazine, pyridine and pyrimidine. Examples of bicylic ring systems Cy1 include, among others, radicals of bicyclo[3.3.0]octane, bicyclo[2.2.1]heptane, bicyclo[2.2.2]octane, bicyclo[4.3.0]nonene, bicyclo[4.4.0]decane, bicyclo[3.3.1]nonene, bicyclo[3.2.1]octane, naphthalene, benzimidazole, benzofuran, benzothiazole, benzothiophene, imidazopyrazine, imidazopyridazine, imidazopyridine, imidazopyrimidine, indazole, indole, isoindole, isoquinoline, tetrahydroisoquinoline, naphthiridine, pyrazolopyrazine, pyrazolopyridine, pyrazolopyrimidine, purine, quinazoline, quinoline and quinoxaline.

The expression “optionally substituted with one or more” means that a group may be unsubstituted or substituted with one or more, preferably with 1, 2, 3 or 4 substituents, provided that this group has 1, 2, 3 or 4 positions susceptible of being substituted.

As used therein the term “treatment” includes treatment, prevention and management of such condition. The term “pharmaceutically acceptable” as used herein refers to those compounds, compositions, and/or dosage forms which are, within the scope of medical judgement, suitable for use in contact with the tissues of humans and animals without excessive toxicity, irritation, allergic response, or other problem or complication, commensurate with a reasonable benefit/risk ratio.

The present invention relates to a process for the preparation of the new compounds previously described as well as derivatives, analogues, tautomeric forms, stereoisomers, polymorphs or pharmaceutically acceptable salts and solvates thereof.

The compounds of the present invention may be synthesized by different routes. They may be prepared by the methods described below, as well as by other standard methods in the field of organic synthesis, or variations thereof obvious to a person skilled in the art, who will understand that the functional groups present in the molecule should be consistent with the described reactions. This fact may require in some cases a modification in the order of the reaction or the choice of one particular method to obtain the desired compound. The use of some of the reactants may require conditions such as the use of anhydrous solvents and inert atmosphere. Moreover, in some of the methods showed below it may be desirable or necessary to protect the functional groups present in the compounds or intermediates of the invention by conventional protecting groups. Many protecting groups as well as procedures for their introduction and removal are described in Greene T. W. and Wuts P. G. M., “Protective Groups in Organic Synthesis”, John Wiley & Sons, 3rd Edition, 1999.

Unless otherwise stated, the meanings of the groups R1, R2, R3, R4, R5, R6, R7 and X are the ones described in the general formula I.

A compound of formula I may be obtained starting form a compound of formula II as shown below:

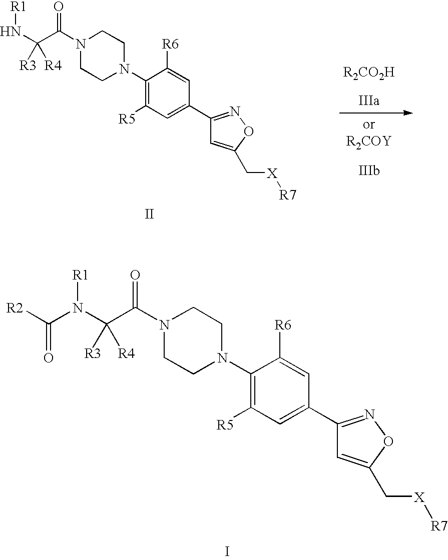

Thus, a compound of formula II may be reacted with a carboxylic acid of formula IIIa in the presence of an activating agent, such as the combination of 1-(3-dimethylaminopropyl)-3-ethylcarbodiimide hydrochloride (EDC) and 1-hydroxybenzotriazole (HOBT) in the presence of a base, such as triethylamine, in a solvent, such as ethyl acetate, N,N-dimethylformamide or tetrahydrofuran, at a temperature between room temperature and the temperature of the boiling point of the solvent. Alternatively, a compound of formula II may be reacted with the corresponding carboxylic acid derivative of formula IIIb, wherein Y represents —CN, —OC(═O)(C₁-C₄)alkyl, —O(C₁-C₄)alkyl, —N[(C₁-C₄)alkyl]₂ or halogen, preferably chloro. This reaction is carried out in the presence of a base such as triethylamine, in a solvent, such as dichloromethane, ethyl acetate or N,N-dimethylformamide and at a temperature between room temperature and the temperature of the boiling point of the solvent.

A compound of formula I may also be obtained by reaction of a compound of formula IV with a compound of formula Va or a compound of formula Vb, wherein Y represents —CN, —OC(═O)(C₁-C₄)alkyl, —O(C₁-C₄)alkyl —N[(C₁-C₄)alkyl]₂ or halogen, preferably chloro, in analogous conditions to those described for the synthesis of I starting from II and IIIa or IIIb, as shown below:

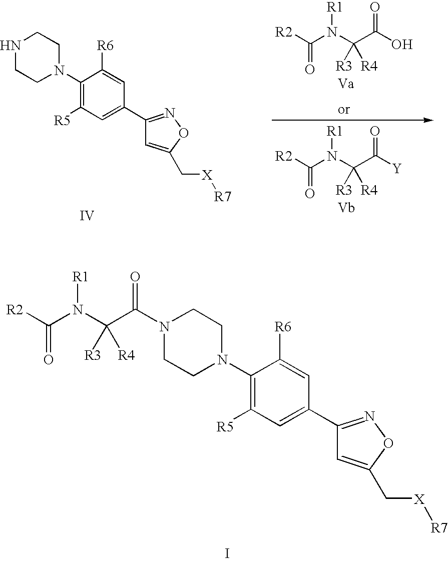

Compounds of formula I wherein R2 represents —NHRb and Rb has the meaning described in general formula I (that is compounds of formula Ia) may also be obtained as shown below:

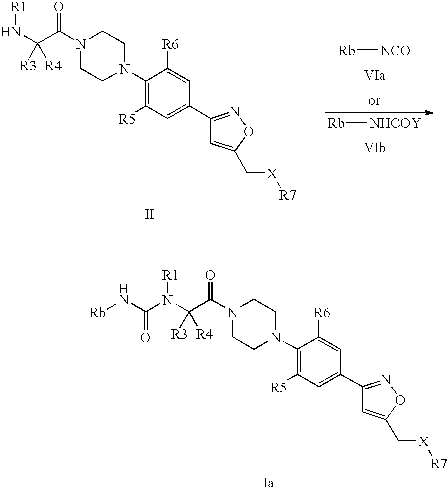

Thus, a compound of formula II may be reacted with an isocyanate of formula VIa in the presence of a solvent, such as N,N-dimethylformamide, preferably at room temperature. Alternatively, a compound of formula II may be reacted with a compound of formula VIb wherein Y represents halogen, preferably chloro, in the presence of a base such as for example triethylamine, in a solvent, such as dichloromethane, ethyl acetate or N,N-dimethylformamide, preferably at room temperature.

Compounds of formula I wherein R2 represents —ORb and Rb has the meaning previously described (that is compounds of formula Ib) may also be obtained by reaction of a compound of formula II with a compound of formula VIIa, wherein Y represents —O-succinimidyl, —OC(═O)(C₁-C₄)alkyl or halogen, preferably chloro.

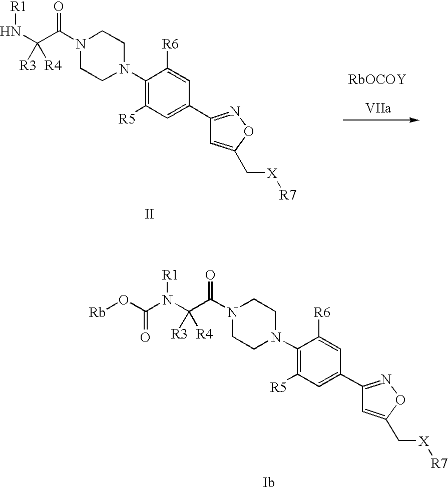

Usually, this reaction is carried out in the presence of a base such as triethylamine, sodium hydroxide or sodium bicarbonate, in a solvent, such as dioxane, water, dichloromethane, tetrahydrofuran, ethyl acetate or N,N-dimethylformamide and at a temperature between room temperature and the temperature of the boiling point of the solvent.

Alternatively the preparation of ureas of formula Ia and carbamates of formula Ib may also be carried out by a sequence of two steps. In a first step an amine of formula II is reacted with a activating agent such as triphosgene or carbonyldiimidazole, in the presence of a base, such as diisopropylethylamine, triethylamine or N-methylmorpholine, in a solvent such as acetonitrile, chloroform, dichloromethane or N,N-dimethylformamide. Then, the resulting compound is reacted with an amine of formula Rb—NH₂ (VIc) (for the ureas) or with an alcohol of formula Rb—OH (VIIb) (for the carbamates) in a solvent, for example the same used in the first step, and at a temperature between room temperature and the temperature of the boiling point of the solvent.

Some compounds of formula I may be converted to other compounds of formula I by reactions well known in the field of organic synthesis, that include but are not limited to the hydrolysis of an ester or the protection/deprotection of a protecting group, among others.

Compounds of formula II may be obtained as shown below:

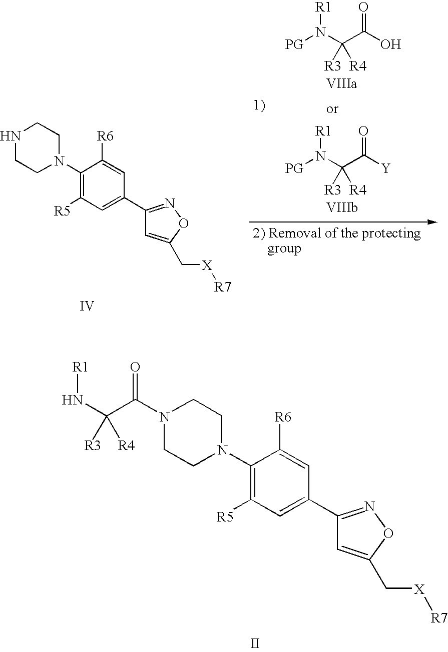

In a first step a compound of formula IV is reacted with a compound of formula VIIIa or a compound of formula VIIIb, wherein PG represents a protecting groupo, such as for example tert-butoxycarbonyl (Boc) or fluorenylmethoxycarbonyl (Fmoc) and Y represents —CN, —OC(═O)(C₁-C₄)alkyl, —OC₁₋₄alkyl, —NRC₁-C₄)alkyl or halogen, preferably chloro, in analogous conditions to those described for the preparation of amides. In a second step the protecting group of the resulting compound is removed following methods described in the literature.

Compounds of formula II wherein R1 represents —H (that is compounds of formula IIa) may also be obtained by a sequence of two steps, as shown below:

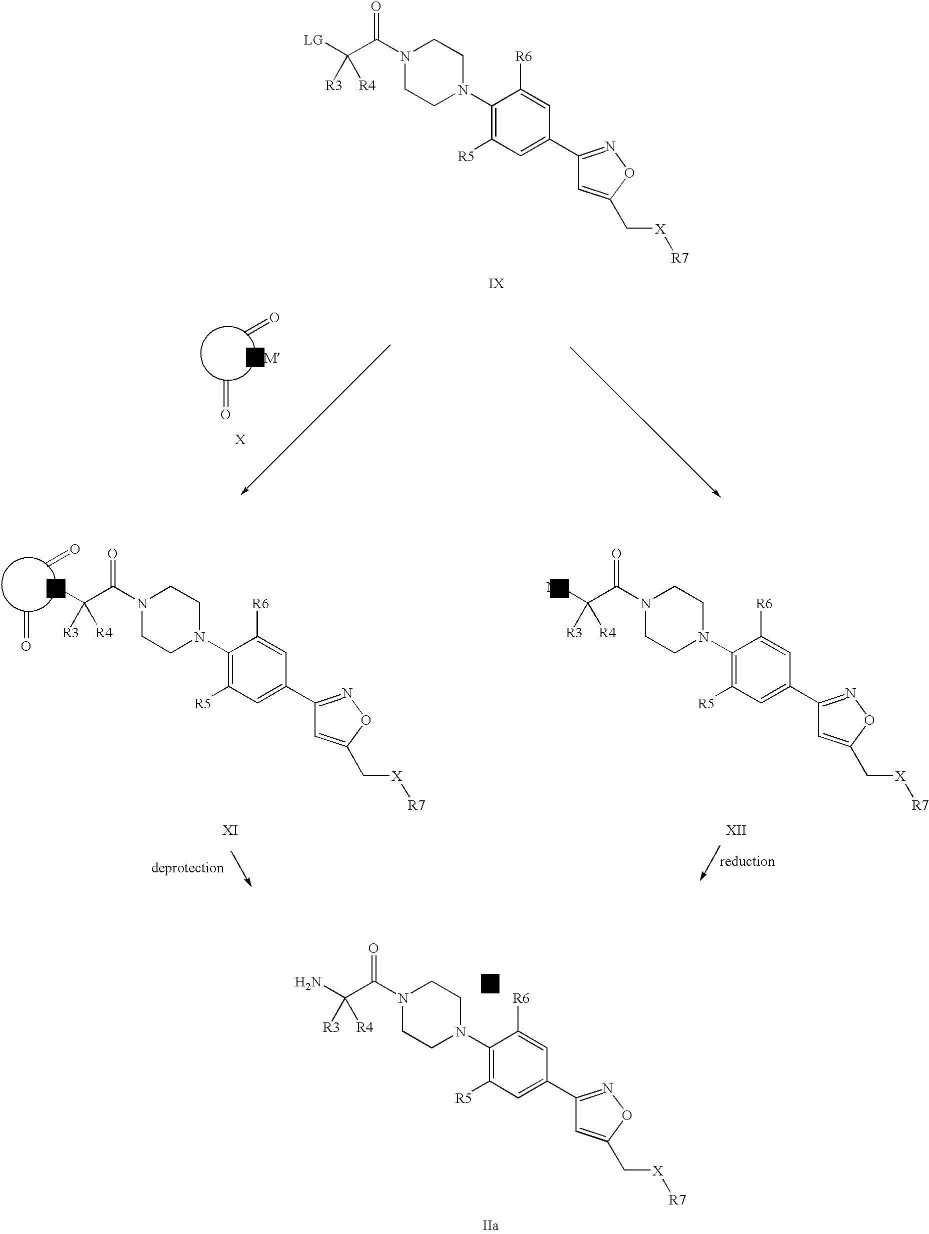

In a first step a compound of formula IX, wherein LG represents halogen, methanesulfonyloxy or p-toluenesulfonyloxy among others is reacted with an azide, such as for example sodium or potassium azide, to give a compound of formula XII. Alternatively a compound of formula IX may be reacted with a compound of formula X, for example potassium phthalimide, to give a compound of formula XI. Both reactions are carried out in a solvent, such as for example N,N-dimethylformamide and preferably heating. Alternatively may be carried out using microwaves. Then compounds of formula XI and XII may be converted into a compound of formula IIa by deprotection and reduction reactions respectively. The deprotection reaction is carried out in the presence of hydrazine, in a solvent such as ethanol or methanol, preferably heating. The reduction reaction is carried out under hydrogen atmosphere, in the presence of a catalyst such as for example Pd—C, in a solvent, such as ethanol, methanol, tetrahydrofuran or ethyl acetate, preferably at room temperature.

Compounds of formula IX may be obtained by reaction of a compound of formula IV and a compound of formula XIIIa or XIIIb, wherein Y represents —CN, —OC(═O)(C₁-C₄)alkyl, —O(C₁-C₄)alkyl —N[(C₁-C₄)alkyl]₂ or halogen, preferably chloro, in analogous conditions to those described for the preparation of compounds of formula I starting from compounds of formula II and compounds of formula IIIa and IIIb respectively.

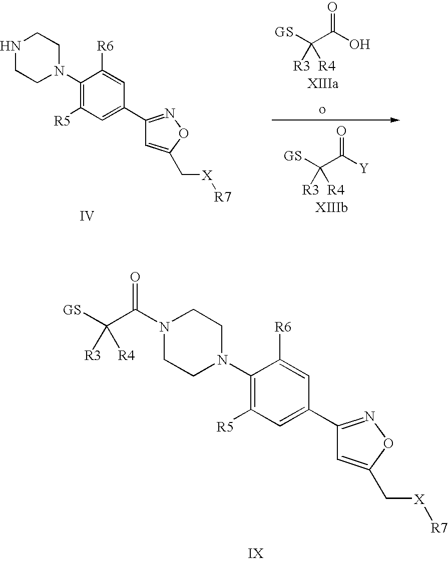

The compounds IIIa, IIIb, Va, Vb, VIa, VIb, VIc, VIIa, VIIb, VIIIa, VIIIb, X, XIIIa and)(Mb are commercially available or may be easily obtained by conventional methods. For example compounds of formula VIIIa and VIIIb may be prepared according to B. S. Furniss “Textbook of practical Organic Chemistry” 5th Ed. (1989) Longman Scientific & Technical. Compounds of formula IV may be obtained as described in WO 03/008395. As it will be obvious for a skilled in the art, some of the reactions previously described may also be carried out on compounds of formula I.

An embodiment of the invention relates to compounds of formula I which are N-oxides. Another embodiment of the invention relates to compounds of formula I wherein Rd represents halogen, ═CRaRc, ═CRcRc, —CN, —C(═O)Re′, —C(═O)ORe′, —C(═O)NRe′Rh′, ═O, —ORe′, —OC(═O)Re′, —NRe′Rh′, ═NRe′, —N⁺Re′Rh′Rh′, —N₃, —NRh′ORe′, —NRe′ORh′, —NO₂, —NRe′C(═O)Rh′, —NRh′C(═O)Re′, —NRe′C(═O)ORh′, —NRh′C(═O)ORe′, —NRe′C(═O)NRe′Rh′ or —NRh′C(═O)NRe′Rh′.

Another embodiment of the invention relates to compounds of formula I wherein R1 represents —H or —(C₁-C₄)alkyl optionally substituted with one or more groups Ra. Another embodiment of the invention relates to compounds of formula I wherein R1 represents —H.

Another embodiment of the invention relates to compounds of formula I wherein R2 represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Rd and/or one group Rf. Another embodiment of the invention relates to compounds of formula I wherein R2 represents -Cy1 optionally substituted with one or more groups independently selected from —Re, halogen, ═CRaRc, ═CRcRc, —CN, —C(═O)Re′, —C(═O)ORe′, —C(═O)NRe′Rh′, ═O, —ORe′, —OC(═O)Re′, —NRe′Rh′, ═NRe′, —NRe′Rh′Rh′, —N₃, —NRh′ORe′, —NRe′ORh′, —NO₂, —NRe′C(═O)Rh′, —NRh′C(═O)Re′, —NRe′C(═O)ORh′, —NRh′C(═O)ORe′, —NRe′C(═O)NRe′Rh′ or —NRh′C(═O)NRe′Rh′. Another embodiment of the invention relates to compounds of formula I wherein R2 is selected from the group consisting of phenyl, a C- or N-radical of an aromatic 5- or 6-membered monocyclic ring containing from one to three heteroatoms independently selected from O, S and N, and a C- or N-radical of an aromatic bicyclic ring system containing from one to three heteroatoms independently selected from O, S and N, that comprises a 5- or 6-membered ring system fused to a 5- or 6-membered ring system, wherein all previous ring systems may be optionally substituted with —(C₁-C₄)alkyl, —(C₂-C₄)alkenyl, —(C₂-C₄)alkynyl, halogen, —CN, —C(═O)Re′, ═O, —ORe′, —NRe′Rh′, —NO₂, —NRe′C(═O)Rh′, —NRh′C(═O)Re′, wherein —(C₁-C₄)alkyl, —(C₂-C₄)alkenyl or —(C₂-C₄)alkynyl may be optionally substituted with one or more groups Ra.

Another embodiment of the invention relates to compounds of formula I wherein R3 represents —H or —(C₁-C₄)alkyl optionally substituted with one or more Ra and R4 represents —H or —(C₁-C₄)alkyl optionally substituted with one or more halogen atoms.

Another embodiment of the invention relates to compounds of formula I wherein R5 represents —F and R6 represents —H or —F. Another embodiment of the invention relates to compounds of formula I wherein X represents —NH— and R7 represents 5- or 6-membered heteroaryl optionally substituted with halogen or Rc. Another embodiment of the invention relates to compounds of formula I wherein X represents —O— and R7 represents —H.

Another embodiment of the invention relates to compounds of formula I wherein R1 represents —H; R2 represents —H, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Rd and/or one group Rf; or R2 represents -Cy1 optionally substituted with one or more groups independently selected from —Re, halogen, ═CRaRc, ═CRcRc, —CN, —C(═O)Re′, —C(═O)ORe′, —C(═O)NRe′Rh′, ═O, —ORe′, —OC(═O)Re′, —NRe′Rh′, ═NRe′, —N⁺Re′Rh′Rh′, —N₃, —NRh′ORe′, —NRe′ORh′, —NO₂, —NRe′C(═O)Rh′, —NRh′C(═O)Re′, —NRe′C(═O)ORh′, —NRh′C(═O)ORe′, —NRe′C(═O)NRe′Rh′ or —NRh′C(═O)NRe′Rh′; R3 represents —H or —(C₁-C₄)alkyl optionally substituted with one or more Ra; R4 represents —H or —(C₁-C₄)alkyl optionally substituted with one or more halogen atoms; R5 represents —F and R6 represents —H or —F; X represents —NH— and R7 represents 5- or 6-membered heteroaryl optionally substituted with halogen or Rc or wherein X represents —O— and R7 represents —H.

Moreover, all possible combinations of the particular embodiments previously mentioned are also part of the application.

The compounds of the present invention may contain one or more basic nitrogen atoms and, therefore, they may form salts with acids, that also form part of this invention. Examples of pharmaceutically acceptable salts include, among others, addition salts with inorganic acids such as hydrochloric, hydrobromic, hydroiodic, nitric, perchloric, sulphuric and phosphoric acid, as well as addition salts of organic acids such as acetic, methanesulfonic, trifluoromethanesulfonic, ethanesulfonic, benzenesulfonic, p-toluenesulfonic, benzoic, camphorsulfonic, mandelic, oxalic, succinic, fumaric, tartaric, and maleic acid. Likewise, compounds of the present invention may contain one or more acid protons and, therefore, they may form salts with bases, that also form part of this invention. Examples of these salts include salts with metal cations, such as for example an alkaline metal ion, an alkaline-earth metal ion or an aluminium ion; or it may be coordinated with an organic or inorganic base. There is no limitation on the type of salt that may be used provided that these are pharmaceutically acceptable. Salts may be synthesized from the parent compound which contains a basic or acidic moiety by conventional chemical methods. Generally, such salts may be prepared by reacting the free acid or base forms of these compounds with a stoichiometric amount of the appropriate base or acid in water or in an organic solvent, such as ether, ethyl acetate, ethanol, isopropanol, or acetonitrile or in a mixture thereof. The compounds of formula I and their salts differ in some physical properties but they are equivalent for the purposes of the present invention.

Some of the compounds of formula I of the present invention may exist as unsolvated as well as solvated forms such as, for example, hydrates or alcohol solvates. The present invention encompasses all such above-mentioned forms which are pharmaceutically active.

Some compounds of formula I may exist as N-oxides of any oxidable nitrogen atom of the cited compounds, this invention comprising all N-oxides of the described compounds.

Some of the compounds of general formula I may exhibit polymorphism, encompassing the present invention all the possible polymorphic forms, and mixtures thereof. Various polymorphs may be prepared by crystallization under different conditions or by heating or melting the compound followed by gradual or fast cooling.

Compounds of formula I of the present invention may comprise one or more chiral centers. Additionally, compounds of formula I of the present invention may have further chiral centres. The present invention includes each one of the possible stereoisomers and mixtures thereof, particularly racemic mixtures thereof. A single enantiomer may be prepared by any of the commonly used processes, for example, by chromatographic separation of the racemic mixture on a stationary chiral phase, by resolution of the racemic mixture by fractional crystallisation techniques of the diastereomeric salts thereof, by chiral synthesis, by enzymatic resolution or by biotransformation. This resolution may be carried out on any chiral synthetic intermediate or on products of general Formula I. Alternatively, any enantiomer of a compound of the general Formula I may be obtained by enantiospecific synthesis using optically pure starting materials or reagents of known configuration. Some of the compounds of the present invention may exist as several diastereoisomers, which may be separated by conventional techniques such as chromatography or fractional crystallization. Some compounds of the present invention may exhibit cis/trans isomers. The present invention includes each of the geometric isomers and its mixtures. The present invention covers all isomers and mixtures thereof (for example racemic mixtures) whether obtained by synthesis and also by physically mixing them. Compounds of formula I have antibiotic activity and therefore useful as active ingredients. Therefore, an aspect of the present invention relates to pharmaceutical compositions that comprise an effective amount of a compound as defined in general formula I and one or more pharmaceutically acceptable excipients.

The present invention further provides for pharmaceutical compositions comprising a compound of formula I or a pharmaceutical salt or solvate thereof together with one or more pharmaceutically acceptable excipients, in either single or multiple doses. The examples of the excipients mentioned below are given by way of illustration only and are not to be construed as limiting the scope of the invention.

The compounds of the present invention may be administered in the form of any pharmaceutical formulation. The pharmaceutical formulation will depend upon the nature of the active compound and its route of administration. Any route of administration may be used, for example such as oral, buccal, pulmonary, topical, parenteral (including subcutaneous, intramuscular, and intravenous), transdermal, ocular (ophthalmic), by inhalation, intranasal, otic, transmucosal, implant or rectal administration.

Solid compositions for oral administration include among others tablets, granulates and hard gelatin capsules, formulated both as immediate release or modified release formulations.

The manufacturing method may be based on a simple mixture, dry granulation, wet granulation or lyophilization of the active compound optionally with excipients such as binding agents, fillers, lubricants, disintegrants, wetting agents, sweetening agents, bioadhesive agents, glidants, release modifiers or osmotic agents.

The tablets may be coated according to methods well-known in the art such as aqueous dispersion coating, solvent-based coating or drying coating. The active compound may also be incorporated by coating onto inert pellets using film-coating agents, plasticizers, opacifiers or antiadherent agents. The active compound may also be incorporated by extrusion and spheronization process, by hot melting pelletization. When a dosage unit form is a capsule, it may contain, in addition to materials of the above type, a liquid carrier such as a fatty oil or wax.

Powders and granulates for the preparation of oral suspensions by the addition of water may be obtained by mixing the active compound with dispersing or wetting agents; suspending agents, anticaking agents, buffering agents and preservatives. Other excipients may also be added, for example sweetening, flavouring and colouring agents.

Alternatively, the compounds of the present invention may be incorporated into oral liquid or semisolid preparations such as emulsions, solutions, dispersions, suspensions, syrups, elixirs or in the form of soft gelatin capsules.

Solutions or suspensions may be prepared in water suitably mixed with a surfactant, if necessary. Dispersions may also be prepared in glycerol, liquid polyethylene glycols and mixtures thereof in oils. These preparations may contain a preservative to prevent the growth of microorganisms. Injectable preparations for parenteral administration comprise sterile solutions, suspensions or emulsions in oily or aqueous vehicles, and may contain coadjuvants, such as suspending, stabilizing, tonicity agents or dispersing agents.

The compound may also be formulated for its topical application. Formulations include creams, lotions, gels, powders, solutions, shampoo preparations, oral paste, mouth wash preparations and patches wherein the compound is dispersed or dissolved in suitable excipients such as antimicrobial preservatives, emulsifying agents, emulsion stabilizers, humectants, skin penetrants, buffering agents, surfactants and thickening agents. Preferably, compounds are administered orally, parenterally or topically.

The compounds of the present invention are especially active against pathogen microorganisms including Gram-positives agents, Gram-negatives agents and mycoplasmas, among others. Thus, the present invention relates to the use of a compound of formula I for the manufacture of a medicament for the treatment and/or prevention of bacterial infections in an animal including a human. Therefore, the present invention also relates to a method for the treatment and/or prevention of bacterial infections in an animal including a human, that comprises administering a compound of formula I.

The effective dosage of active ingredient may vary depending on the particular compound administered, the route of administration, the nature and severity of the disease to be treated, as well as the age, the general condition and body weight of the patient, among other factors. A representative example of a suitable dosage range is from about 0.001 to about 100 mg/kg body weight per day, which may be administered as a single or divided doses. However, the dosage administered will be generally left to the discretion of the physician.

Throughout the description and claims the word “comprise” and variations of the word, such as “comprising”, are not intended to exclude other additives, components, elements or steps. The present invention will be further illustrated by the following examples. The examples are given by way of illustration only and are not to be construed as limiting the scope of the invention.

### EXAMPLES

¹H-NMR spectra of the compounds have been recorded using a VARIAN UNITY-300 or MERCURY 400 MHz equipment and chemical shifts are expressed as ppm (δ) from the internal reference trimethylsilane.

Mass spectra have been obtained with an Agilent 1100 VL mass spectrometer.

HPLC-ESI-MS spectra have been performed using the following chromatographic equipment: Agilent model 1000, equipped with a selective mass detector model 1100 VL (atmospheric pressure ionisation with positive ion detection), autosampler, ChemStation software and a laser and using the following chromatographic methods:

Method A: Column Kromasil 100 C18, 40×4.0 mm, 3.5 μm, flow: 0.7 mL/min, eluent: A=0.1% formic acid in water, B=0.1% formic acid in acetonitrile, gradient: 0 min 5% B-8 min 90% B.

Method B: Column Gemini 5u C18 110, 40×4.0 mm, flow: 0.7 mL/min, eluent: A=0.1% formic acid in water, B=0.1% formic acid in acetonitrile, gradient: 0 min 5% B-8 min 90% B.

Unless otherwise stated the HPLC-ESI-MS data indicated in the tables below was obtained using method A.

The following abbreviations have been used in the examples:

DMAP: 4-dimethylaminopyridine

DMF: N,N-dimethylformamide

EDC: 1-(3-dimethylaminopropyl)-3-ethylcarbodiimide hydrochloride

eq: molar equivalent

EtOAc: ethyl acetate

HOBt: 1-hydroxybenzotriazole

HPLC-ESI-MS: high resolution liquid chromatography—electrospray

ionization—mass spectrometry

m/z: relationship mass/charge

rt: retention time

THF: tetrahydrofuran

### Examples of Intermediates of Formula IV:

Compound IV_{—}1 [3-(3-fluoro-4-piperazin-1-ylphenyl)isoxazol-5-ylmethyl]isoxazol-3-ylamine corresponds to the intermediate 10 of patent WO 03/008395 and its synthesis was carried out as described in page 38.

Compound IV_{—}2 N-[3-(3-fluoro-4-piperazin-1-ylphenyl)isoxazol-5-ylmethyl]acetamide was prepared in analogous form to the intermediate IV_{—}1 replacing isoxazol-3-yl-[3-(3,4-difluorophenyl)isoxazol-5-ylmethyl]amine by N-[3-(3,4-difluorophenyl)isoxazol-5-ylmethyl]acetamide (intermediate 9 patent WO 03/008395).

Compound IV_{—}3 [3-(3,5-difluoro-4-piperazin-1-ylphenyl)isoxazol-5-ylmethyl]isoxazol-3-ylamine corresponds to the intermediate 18 of patent WO 03/008395 and its synthesis was carried out as described in page 40.

Compound IV_{—}4 [3-(3-fluoro-4-piperazin-1-ylphenyl)isoxazol-5-yl]methanol corresponds to the intermediate 3 of patent WO 03/008395 and its synthesis was carried out as described in page 34.

### Examples of Intermediates of Formula IX:

Compounds of formula IX shown in table 1 were obtained by one of the following methods.

METHOD 1: To a solution 0.15 M of a carboxylic acid of formula XIIIa (1 eq) in DMF, EDC (1.5 eq), HOBt (1.5 eq) and triethylamine (2 eq) were added. The mixture was stirred for 15 minutes at room temperature. Then, an amine of formula IV (1 eq) was added and the mixture was stirred for 14 hours. Water in an amount of about 10 parts by volume of DMF was added and the precipitate obtained was filtered and washed thoroughly with water. In case that no precipitate was formed, the mixture was extracted three times with EtOAc and then, the organic phases were washed twice with brine, dried over anhydrous sodium sulfate, filtered and concentrated at reduced pressure. If necessary, the obtained product was purified by column chromatography on silica gel.

METHOD 2: To a 0.1 M solution of a compound of formula IV (1 eq) in DMF, triethylamine (1.1 eq), DMAP (0.1 eq) and an acyl chloride of formula XIIIb (1.1 eq) were added. The reaction was followed by thin-layer chromatography until the starting material disappeared. Water in an amount of about 10 parts by volume of DMF was added and the precipitate obtained was filtered and washed thoroughly con water. In case that no precipitate was formed, the mixture was extracted three times with EtOAc and then the organic phases were washed twice with brine, dried over anhydrous sodium sulfate, filtered and concentrated at reduced pressure. If necessary, the obtained product was purified by column chromatography on silica gel.

TABLE 1

| Ex. | Name | Starting materials | HPLC-ESI-MS |
|---|---|---|---|
| IX_1 | 2-Bromo-1-(4-{2-fluoro-4-[5-(isoxazol- | IV_1 and 2-bromo- | rt: 6.370 |
| | 3-ylaminomethyl)isoxazol-3- | propanoic acid (XIIIa_1) | m/z: 478/480 |
| | yl]phenyl}piperazin-1-yl)propan-1-one | | |
| IX_2 | 2-Chloro-1-(4-{2-fluoro-4-[5-(isoxazol- | IV_3 and chloroacetyl | rt: 6.016 |
| | 3-ylaminomethyl)isoxazol-3- | chloride (XIIIb_1) | m/z: 438/440 |
| | yl]phenyl}piperazin-1-yl)ethanone | | |

### Examples of Intermediates of Formula IIIa:

The following intermediates of formula IIIa shown in table 2 were prepared following the four-step synthesis described in P. L. Beaulieu, J. Med. Chem. 2004, 47 (27), 6884 with a slight modification in the last step as described in M. A. Phillips, J. Chem. Soc. 1929, 2820.

TABLE 2

| Ex. | Name | Starting materials | HPLC-ESI-MS |
|---|---|---|---|
| IIIa_68 | 2-Methylbenzimidazole-5- | Acetic acid, methyl 4-chloro-3- | rt: — |
| | carboxylic acid | nitrobenzoate and benzylamine | m/z: 177 |
| IIIa_69 | 1,2-Dimethylbenzimidazole-5- | Acetic acid, methyl 4-chloro-3- | rt: 0.846 |
| | carboxylic acid | nitrobenzoate and methylamine | m/z: 191 |
| IIIa_70 | 1-Cyclopropylmethyl-2- | Acetic acid, methyl 4-chloro-3- | rt: 2.962 |
| | methylbenzimidazole-5- | nitrobenzoate and | m/z: 231 |
| | carboxylic acid | cyclopropylmethylamine | |
| IIIa_71 | 2-Methyl-1- | Acetic acid, methyl 4-chloro-3- | rt: 2.816 |
| | propylbenzimidazole-5- | nitrobenzoate of methyl and | m/z: 219 |
| | carboxylic acid | propylamine | |
| IIIa_72 | 2-Methyl-1-(2- | Acetic acid, 4-chloro-3- | rt: 3.307 |
| | propynyl)benzimidazole-5- | nitrobenzoate and 2- | m/z: 215 |
| | carboxylic acid | propynylamine | |
| IIIa_73 | 1-Allyl-2-methylbenzimidazole- | Acetic acid, methyl 4-chloro-3- | rt: 2.646 |
| | 5-carboxylic acid | nitrobenzoate and allylamine | m/z: 217 |
| IIIa_74 | 1-Cyclopentyl-2-methyl- | Acetic acid, methyl 4-chloro-3- | rt: 3.467 |
| | benzimidazole-5-carboxylic | nitrobenzoate and | m/z: 245 |
| | acid | cyclopentylamine | |
| IIIa_75 | 1-Cyclohexyl-2-methyl- | Acetic acid, methyl 4-chloro-3- | rt: 3.939 |
| | benzimidazole-5-carboxylic | nitrobenzoate and | m/z: 259 |
| | acid | cyclohexylamine | |
| IIIa_137 | 6-(N-Ethyl-N-methyl)amino- | Ethyl 6-chloropyridine-3- | rt: 1.330 |
| | pyridine-3-carboxylic acid | carboxyate and | m/z: 181 |
| | | ethylmethylamine | |
| IIIa_138 | 6-(N,N-dimethyl)aminopyridine- | Ethyl 6-chloropyridine-3- | rt: — |
| | 3-carboxylic acid | carboxyate and dimethylamine | m/z: 167 |
| IIIa_139 | 6-[N-(2-methoxy)ethylamino]- | Ethyl 6-chloropyridine-3- | rt: — |
| | pyridine-3-carboxylic acid | carboxyate and 2- | m/z: 197 |
| | | methoxyethylamine | |
| IIIa_140 | 6-(N-methylamino)pyridine-3- | Ethyl 6-chloropyridine-3- | rt: — |
| | carboxylic acid | carboxyate and methylamine | m/z: 153 |
| IIIa_141 | Hydroxypyridin-3-ylacetic acid | pyridine-3-carbaldehyde and | rt: 0.443 |
| | | potassium cyanide | m/z: 154 |

### Examples of Intermediates of Formula II:

Compounds of formula II shown in table 3 were obtained by one of the methods 1-2 described below.

METHOD 1: Corresponds to a sequence of 2 steps. The first step corresponds to the method 1 described for the preparation of compounds of formula IX, using as starting materials an amine of formula IV and an acid of formula VIIIa.

Then, when PG represents tert-butoxycarbonyl the resulting product was dissolved in ethanol to give a 0.1 M solution and para-toluenesulfonic acid monohydrate (1.5 eq) was added. The reaction was stirred at reflux until the starting material disappeared on thin-layer chromatography. The resulting mixture was concentrated under reduced pressure. An aqueous solution sodium bicarbonate was added to the crude and the mixture extracted three times with EtOAc. Then the organic phases were washed twice with brine, dried over anhydrous sodium sulfate, filtered and concentrated at reduced pressure. If necessary, the obtained product was purified by column chromatography on silica gel.

When PG represents N-(9-fluorenylmethoxycarbonyl) the resulting product was dissolved in THF:DMF 9:1 to give a 0.1 M solution and piperidine (5 eq) was added. The reaction was stirred at room temperature reflux until the starting material disappeared on thin-layer chromatography. THF was removed by evaporation under reduced pressure. An aqueous solution sodium bicarbonate was added to the crude and the mixture extracted three times with EtOAc. Then the organic phases were washed twice with brine, dried over anhydrous sodium sulfate, filtered and concentrated at reduced pressure. If necessary, the obtained product was purified by column chromatography on silica gel.

METHOD 2: Corresponds to a sequence of 2 steps. In the first step, to a solution 0.5 M of a compound of formula IX (1 eq) in dried DMF in a closed-vessel, sodium azide (1.1 eq) was added. The mixture was heated in a microwave oven with simultaneous cooling (150 W; 150° C. until the starting material disappeared on thin-layer chromatography. Water in an amount of about 10 parts by volume of DMF was added at room temperature and the mixture was stirred. The obtained precipitate was filtered and washed thoroughly with water. In case that no precipitate was formed, the mixture was extracted three times with EtOAc and then the organic phases were washed twice with brine, dried over anhydrous sodium sulfate, filtered and concentrated at reduced pressure. If necessary, the obtained product was purified by column chromatography on silica gel.

The resulting compound of formula XII was dissolved in methanol to give a 0.1 M solution and Pd—C at 10% (10% by weight of the product obtained in the first step) was added. The suspension was stirred under hydrogen atmosphere until the starting material disappeared on thin-layer chromatography. The mixture was filtered through celite and the filtrate was concentrated by evaporation under reduced pressure. If necessary, the obtained product was purified by column chromatography on silica gel.

TABLE 3

| Ex. | Name | Starting materials | HPLC-ESI-MS |
|---|---|---|---|
| II_1 | 2-Amino-1-(4-{2-fluoro-4-[5-(isoxazol-3- | IV_1 and N-tert- | rt: 3.677 |
| | ylaminomethyl)isoxazol-3- | butoxycarbonylglycine | m/z: 401 |
| | yl]phenyl}piperazin-1-yl)ethanone | (VIIIa_1) | |
| II_2 | 2-Amino-1-(4-{2-fluoro-4-[5-(isoxazol-3- | IX_1 and sodium azide | rt: 3.895 |
| | ylaminomethyl)isoxazol-3- | | m/z: 415 |
| | yl]phenyl}piperazin-1-yl)propanone | | |
| II_3 | 2-Amino-1-(4-{2-fluoro-4-[5-(isoxazol-3- | IV_1 and N-tert-butoxy- | rt: 4.467 |
| | ylaminomethyl)isoxazol-3-yl]phenyl}- | carbonyl-DL-α- | m/z: 477 |
| | piperazin-1-yl)-2-phenylethanone | phenylglycine (VIIIa_2) | |
| II_4 | N-(3-{4-[4-(2-Aminoacetyl)piperazin-1- | IV_2 and N-tert-butoxy- | rt: 3.147 |
| | yl]-3-fluorophenyl}isoxazol-5- | carbonylglycine | m/z: 376 |
| | ylmethyl)acetamide | (VIIIa_1) | |
| II_5 | 1-(4-{2-Fluoro-4-[5-(isoxazol-3- | IV_1 and N-(9-fluorenylmethoxycarbonyl)- | rt: 7.428 |
| | ylaminomethyl)isoxazol-3-yl]phenyl}- | N- | m/z: 415 |
| | piperazin-1-yl)-2-methylaminoethanone | methylglycine (VIIIa_3) | |
| II_6 | 2-Amino-1-(4-{2,6-difluoro-4-[5- | IX_2 and sodium azide | rt: 3.942 |
| | (isoxazol-3-ylaminomethyl)isoxazol-3- | | m/z: 419 |
| | yl]phenyl}piperazin-1-yl)ethanone | | |
| II_7 | (S)-2-Amino-1-(4-{2-fluoro-4-[5- | IV_1 and N-tert- | rt: 3.941 |
| | (isoxazol-3-ylaminomethyl)isoxazol-3- | butoxycarbonyl-L- | m/z: 415 |
| | yl]phenyl}piperazin-1-yl)propanone | alanine (VIIIa_4) | |
| II_8 | (R)-2-Amino-1-(4-{2-fluoro-4-[5- | IV_1 and N-tert- | rt: 3.950 |
| | (isoxazol-3-ylaminomethyl)isoxazol-3- | butoxycarbonyl-D- | m/z: 415 |
| | yl]phenyl}piperazin-1-yl)propanone | alanine (VIIIa_5) | |
| II_9 | (R)-2-Amino-1-(4-{2-fluoro-4-[5- | IV_1 and N-tert- | rt: 4.317 |
| | (isoxazol-3-ylaminomethyl)isoxazol-3- | butoxycarbonyl-D- | m/z: 443 |
| | yl]phenyl}piperazin-1-yl)-3-methyl- | valine (VIIIa_6) | |
| | butanone | | |
| II_10 | (S)-2-Amino-1-(4-{2-fluoro-4-[5- | IV_1 and N-tert- | rt: 4.338 |
| | (isoxazol-3-ylaminomethyl)isoxazol-3- | butoxycarbonyl-L-valine | m/z: 443 |
| | yl]phenyl}piperazin-1-yl)-3- | (VIIIa_7) | |
| | methylbutanone | | |
| II_011 | 2-Amino-1-{4-[4-(5-hydroxymethyl- | IV_4 and VIIIa_1 (N- | rt: 3.146 |
| | isoxazol-3-yl)phenyl]piperazin-1- | tert-butoxycarbonyl- | m/z: 335 |
| | yl}ethanone | glycine) | |
| II_012 | 2-Amino-1-(4-{2-fluoro-4-[5-(isoxazol-3- | IV_001 and VIIIa_8 | rt: 4.026 |
| | ylaminomethyl)isoxazol-3-yl]phenyl}- | (Boc-alfa-methyl- | m/z: 429 |
| | piperazin-1-yl)2-methylpropanone | alanine) | |
| II_013 | (1-Aminocyclopentyl)-(4-{4-[5-(isoxazol- | IV_001 and VIIIa_9 (1- | rt: 4.275 |
| | 3-ylaminomethyl)isoxazol-3-yl]phenyl}- | (N-Boc-amino)cyclo- | m/z: 455 |
| | piperazin-1-yl)methanone | pentane carboxylic | |
| | | acid) | |
| II_014 | (S)-2-Amino-1-{4-[4-(5-hydroxy- | IV_004 and VIIIa_4 (N- | rt: 3.384 |
| | methylisoxazol-3-yl)phenyl]piperazin-1- | tert-butoxycarbonyl-L- | m/z: 349 |
| | yl}propan-1-one | alanine) | |

### Examples of Compounds of Formula I:

Compounds of formula I shown in table 4 were obtained by one of the methods 1-4 described below.

METHOD 1: Corresponds to the method 1 described for the preparation of compounds of formula IX, using as starting materials an amine of formula II and an acid of formula IIIa.

METHOD 2: Corresponds to the method 2 described for the preparation of compounds of formula IX, using as starting materials an amine of formula II and an acyl chloride of formula IIIb.

METHOD 3: To a solution of a compound of formula I (1 eq) wherein R2 represents alkyl substituted with —OC(═O)Rc, wherein Rc represents alkyl or aryl, in a mixture THF:methanol:water 4:1:1 to give a 0.1 M solution, a solution 1 N of sodium hydroxide (1.1 eq) was added. The reaction was stirred at room temperature until the starting material disappeared on thin-layer chromatography. The resulting mixture was concentrated by evaporation under reduced pressure. Water was added to the crude and the mixture was extracted three times with dichloromethane and then the organic phases were washed twice with brine, dried over anhydrous sodium sulfate, filtered and concentrated under reduced pressure, to yield the corresponding alcohol of formula I.

METHOD 4: A compound I comprising a tert-butoxycarbonylamino group (1 eq) was dissolved in dichloromethane to give a 0.1 M solution. Trifluoroacetic acid (20 eq) was added and the reaction was stirred at room temperature until the starting material disappeared on thin-layer chromatography. The resulting mixture was concentrated by evaporation under reduced pressure. An aqueous solution of sodium bicarbonate was added to the crude and the mixture was extracted three times with dichloromethane. Then, the organic phases were washed twice with brine, dried over anhydrous sodium sulfate, filtered and concentrated under reduced pressure. The obtained product was purified by column chromatography on silica gel, to yield the corresponding amine of formula I.

TABLE 4

| Ex. | Name | Starting materials | HPLC-ESI-MS |
|---|---|---|---|
| I_1 | N-[2-(4-{2-Fluoro-4-[5-(isoxazol-3- | II_1 and N,N- | rt: 4.546 |
| | ylaminomethyl)isoxazol-3-yl]phenyl}piperazin- | dimethylformamide | m/z: 429 |
| | 1-yl)-2-oxoethyl]formamide | (IIIb_1) | |
| I_2 | N-[2-(4-{2-Fluoro-4-[5-(isoxazol-3- | II_1 and acetyl | rt: 4.588 |
| | ylaminomethyl)isoxazol-3-yl]phenyl}piperazin- | chloride (IIIb_2) | m/z: 443 |
| | 1-yl)-2-oxoethyl]acetamide | | |
| I_3 | N-[2-(4-{2-Fluoro-4-[5-(isoxazol-3- | II_1 and cyclopentanecarbonylchloride | rt: 5.757 |
| | ylaminomethyl)isoxazol-3-yl]phenyl}piperazin- | (IIIb_3) | m/z: 497 |
| | 1-yl)-2-oxoethyl]cyclopentanecarboxamide | | |
| I_4 | N-[2-(4-{2-Fluoro-4-[5-(isoxazol-3- | II_1 and | rt: 4.391 |
| | ylaminomethyl)isoxazol-3-yl]phenyl}piperazin- | thiazolidine-5- | m/z: 516 |
| | 1-yl)-2-oxoethyl]thiazolidine-5-carboxamide | carboxylic acid | |
| | | (IIIa_1) | |
| I_5 | N-[2-(4-{2-Fluoro-4-[5-(isoxazol-3- | II_1 and | rt: 4.942 |
| | ylaminomethyl)isoxazol-3-yl]phenyl}piperazin- | acetoxyacetyl | m/z: 501 |
| | 1-yl)-2-oxoethylcarbamoyl]methyl acetate | chloride (IIIb_4) | |
| I_6 | N-[2-(4-{2-Fluoro-4-[5-(isoxazol-3- | II_1 and | rt: 4.905 |
| | ylaminomethyl)isoxazol-3-yl]phenyl}piperazin- | methoxyacetyl | m/z: 473 |
| | 1-yl)-2-oxoethyl]-2-methoxyacetamide | chloride (IIIb_5) | |
| I_7 | N-[2-(4-{2-Fluoro-4-[5-(isoxazol-3- | I_5 | rt: 4.446 |
| | ylaminomethyl)isoxazol-3-yl]phenyl}piperazin- | | m/z: 459 |
| | 1-yl)-2-oxoethyl]-2-hydroxyacetamide | | |
| I_8 | tert-Butyl N-{[2-(4-{2-fluoro-4-[5-(isoxazol-3- | II_1 and N-tert- | rt: 5.611 |
| | ylaminomethyl)isoxazol-3-yl]phenyl}piperazin- | butoxycarbonylglycine | m/z: Not |
| | 1-yl)-2-oxoethylcarbamoyl]methyl}carbamate | (IIIa_2) | detected |
| I_9 | N-[2-(4-{2-Fluoro-4-[5-(isoxazol-3- | II_1 and 3- | rt: 4.289 |
| | ylaminomethyl)isoxazol-3-yl]phenyl}piperazin- | ureidopropionic | m/z: 515 |
| | 1-yl)-2-oxoethyl]-3-ureidopropionamide | acid (IIIa_3) | |
| I_10 | 2-(2,5-Dioxoimidazolidin-4-yl)-N-[2-(4-{2-fluoro- | II_1 and 5- | rt: 4.363 |
| | 4-[5-(isoxazol-3-ylaminomethyl)isoxazol-3- | hydantoinacetic | m/z: 541 |
| | yl]phenyl}piperazin-1-yl)-2-oxoethyl]acetamide | acid (IIIa_4) | |
| I_11 | 2-(2,6-Dioxo-1,2,3,6-tetrahydropyrimidin-4-yl)- | II_1 and 4- | rt: 4.330 |
| | N-[2-(4-{2-fluoro-4-[5-(isoxazol-3- | uracylacetic acid | m/z: 553 |
| | ylaminomethyl)isoxazol-3-yl]phenyl}piperazin- | (IIIa_5) | |
| | 1-yl)-2-oxoethyl]acetamide | | |
| I_12 | N-[2-(4-{2-Fluoro-4-[5-(isoxazol-3- | II_1 and 1- | rt: 4.653 |
| | ylaminomethyl)isoxazol-3-yl]phenyl}ipiperazin- | (aminocarbonyl)-1- | m/z: 512 |
| | 1-yl)-2-oxoethyl]cyclopropane-1,1- | cyclopropanecarboxylic | |
| | dicarboxamide | acid (IIIa_6) | |
| I_13 | N-[2-(4-{2-Fluoro-4-[5-(isoxazol-3- | II_1 and 2-nitrobenzoyl | rt: 5.650 |
| | ylaminomethyl)isoxazol-3-yl]phenyl}piperazin- | chloride | m/z: 550 |
| | 1-yl)-2-oxoethyl]-2-nitrobenzamide | (IIIb_6) | |
| I_14 | N-[2-(4-{2-Fluoro-4-[5-(isoxazol-3- | II_1 and 4- | rt: 5.789 |
| | ylaminomethyl)isoxazol-3-yl]phenyl}piperazin- | methoxybenzoyl | m/z: 535 |
| | 1-yl)-2-oxoethyl]-4-methoxybenzamide | chloride (IIIb_7) | |
| I_15 | N-[2-(4-{2-Fluoro-4-[5-(isoxazol-3- | II_1 and 3- | rt: 5.210 |
| | ylaminomethyl)isoxazol-3-yl]phenyl}piperazin- | hydroxybenzoic | m/z: 521 |
| | 1-yl)-2-oxoethyl]-3-hydroxybenzamide | acid (IIIa_7) | |
| I_16 | N-[2-(4-{2-Fluoro-4-[5-(isoxazol-3- | II_1 and 4- | rt: 5.096 |
| | ylaminomethyl)isoxazol-3-yl]phenyl)piperazin- | hydroxybenzoic | m/z: 521 |
| | 1-yl)-2-oxoethyl]-4-hydroxybenzamide | acid (IIIa_8) | |
| I_17 | N-[2-(4-{2-Fluoro-4-[5-(isoxazol-3- | II_1 and 2- | rt: 6.038 |
| | ylaminomethyl)isoxazol-3-yl]phenyl}piperazin- | hydroxybenzoic | m/z: 521 |
| | 1-yl)-2-oxoethyl]-2-hydroxybenzamide | acid (IIIa_9) | |
| I_18 | N-[2-(4-{2-Fluoro-4-[5-(isoxazol-3- | II_1 and 2-hydroxy- | rt: 6.395 |
| | ylaminomethyl)isoxazol-3-yl]phenyl}piperazin- | 4-methylbenzoic | m/z: 535 |
| | 1-yl)-2-oxoethyl]-2-hydroxy-4- | acid (IIIa_10) | |
| | methylbenzamide | | |
| I_19 | N-[2-(4-{2-Fluoro-4-[5-(isoxazol-3- | II_1 and 2-hydroxy- | rt: 6.752 |
| | ylaminomethyl)isoxazol-3-yl]phenyl}piperazin- | 3-methylbenzoic | m/z: 535 |
| | 1-yl)-2-oxoethyl]-2-hydroxy-3- | acid (IIIa_11) | |
| | methylbenzamide | | |
| I_20 | 4-Fluoro-N-[2-(4-{2-fluoro-4-[5-(isoxazol-3- | II_1 and 4-fluoro-2- | rt: 6.313 |
| | ylaminomethyl)isoxazol-3-yl]phenyl}piperazin- | hydroxybenzoic | m/z: 539 |
| | 1-yl)-2-oxoethyl]-2-hydroxybenzamide | acid (IIIa_12) | |
| I_21 | 5-Fluoro-N-[2-(4-{2-fluoro-4-[5-(isoxazol-3- | II_1 and 5-fluoro-2- | rt: 6.116 |
| | ylaminomethyl)isoxazol-3-yl]phenyl}piperazin- | hydroxybenzoic | m/z: 539 |
| | 1-yl)-2-oxoethyl]-2-hydroxybenzamide | acid (IIIa_13) | |
| I_22 | 3-Fluoro-N-[2-(4-{2-fluoro-4-[5-(isoxazol-3- | II_1 and 3-fluoro-4- | rt: 5.267 |
| | ylaminomethyl)isoxazol-3-yl]phenyl}piperazin- | hydroxybenzoic | m/z: 539 |
| | 1-yl)-2-oxoethyl]-4-hydroxybenzamide | acid (IIIa_14) | |
| I_23 | 2-Fluoro-N-[2-(4-{2-fluoro-4-[5-(isoxazol-3- | II_1 and 2-fluoro-6- | rt: 6.715 |
| | ylaminomethyl)isoxazol-3-yl]phenyl}piperazin- | hydroxybenzoic | m/z: 539 |
| | 1-yl)-2-oxoethyl]-6-hydroxybenzamide | acid (IIIa_15) | |
| I_24 | N-[2-(4-{2-Fluoro-4-[5-(isoxazol-3- | II_1 and 3-hydroxy- | rt: 5.835 |
| | ylaminomethyl)isoxazol-3-yl]phenyl}piperazin- | 2,4,5- | m/z: 575 |
| | 1-yl)-2-oxoethyl]-3-hydroxy-2,4,5- | trifluorobenzoic | |
| | trifluorobenzamide | acid (IIIa_16) | |
| I_25 | 2,3-Dihydroxy-N-[2-(4-{2-fluoro-4-[5-(isoxazol- | II_1 and 2,3- | rt: 5.487 |
| | 3-ylaminomethyl)isoxazol-3-yl]phenyl}- | dihydroxybenzoic | m/z: 537 |
| | piperazin-1-yl)-2-oxoethyl]benzamide | acid (IIIa_17) | |
| I_26 | 3,4-Dihydroxy-N-[2-(4-{2-fluoro-4-[5-(isoxazol- | II_1 and 3,4- | rt: 4.870 |
| | 3-ylaminomethyl)isoxazol-3-yl]phenyl}- | dihydroxybenzoic | m/z: 537 |
| | piperazin-1-yl)-2-oxoethyl]benzamide | acid (IIIa_18) | |
| I_27 | 2,6-Dihydroxy-N-[2-(4-{2-fluoro-4-[5-(isoxazol- | II_1 and 2,6- | rt: 5.968 |
| | 3-ylaminomethyl)isoxazol-3-yl]phenyl}- | dihydroxybenzoic | m/z: 537 |
| | piperazin-1-yl)-2-oxoethyl]benzamide | acid (IIIa_19) | |
| I_28 | 2,4-Dihydroxy-N-[2-(4-{2-fluoro-4-[5-(isoxazol- | II_1 and 2,4- | rt: 5.411 |
| | 3-ylaminomethyl)isoxazol-3-yl]phenyl}- | dihydroxybenzoic | m/z: 537 |
| | piperazin-1-yl)-2-oxoethyl]benzamide | acid (IIIa_20) | |
| I_29 | N-[2-(4-{2-Fluoro-4-[5-(isoxazol-3-ylamino- | II_1 and 2-hydroxy- | rt: 5.983 |
| | methyl)isoxazol-3-yl]phenyl}piperazin-1-yl)-2- | 5-methoxybenzoic | m/z: 551 |
| | oxoethyl]-2-hydroxy-5-methoxybenzamide | acid (IIIa_21) | |
| I_30 | N-[2-(4-{2-Fluoro-4-[5-(isoxazol-3-ylamino- | II_1 and 2-hydroxy- | rt: 6.168 |
| | methyl)isoxazol-3-yl]phenyl}piperazin-1-yl)-2- | 4-methoxybenzoic | m/z: 551 |
| | oxoethyl]-2-hydroxy-4-methoxybenzamide | acid (III_a22) | |
| I_31 | N-[2-(4-{2-Fluoro-4-[5-(isoxazol-3- | II_1 and 3-hydroxy- | rt: 5.760 |
| | ylaminomethyl)isoxazol-3-yl]phenyl}piperazin- | 4-nitrobenzoic acid | m/z: 566 |
| | 1-yl)-2-oxoethyl]-3-hydroxy-4-nitrobenzamide | (IIIa_23) | |
| I_32 | 4-Amino-N-[2-(4-{2-fluoro-4-[5-(isoxazol-3- | II_1 and 4-amino-2- | rt: 5.354 |
| | ylaminomethyl)isoxazol-3-yl]phenyl}piperazin- | hydroxybenzoic | m/z: 536 |
| | 1-yl)-2-oxoethyl]-2-hydroxybenzamide | acid (IIIa_24) | |
| I_33 | 5-Amino-N-[2-(4-{2-fluoro-4-[5-(isoxazol-3- | II_1 and 5-amino-2- | rt: 4.363 |
| | ylaminomethyl)isoxazol-3-yl]phenyl}piperazin- | hydroxybenzoic | m/z: 536 |
| | 1-yl)-2-oxoethyl]-2-hydroxybenzamide | acid (IIIa_25) | |
| I_34 | 2-Amino-N-[2-(4-{2-fluoro-4-[5-(isoxazol-3- | II_1 and 2-amino-5- | rt: 4.362 |
| | ylaminomethyl)isoxazol-3-yl]phenyl}piperazin- | hydroxybenzoic | m/z: 536 |
| | 1-yl)-2-oxoethyl]-5-hydroxybenzamide | acid (IIIa_26) | |
| I_35 | 4-Amino-N-[2-(4-{2-fluoro-4-[5-(isoxazol-3- | II_1 and 4-amino-3- | rt: 4.749 |
| | ylaminomethyl)isoxazol-3-yl]phenyl}piperazin- | hydroxybenzoic | m/z: 536 |
| | 1-yl)-2-oxoethyl]-3-hydroxybenzamide | acid (IIIa_27) | |
| I_36 | 3-Amino-N-[2-(4-{2-fluoro-4-[5-(isoxazol-3- | II_1 and 3-amino-4- | rt: 4.398 |
| | ylaminomethyl)isoxazol-3-yl]phenyl}piperazin- | hydroxybenzoic | m/z: 536 |
| | 1-yl)-2-oxoethyl]-4-hydroxybenzamide | acid (IIIa_28) | |
| I_37 | 4-Amino-N-[2-(4-{2-fluoro-4-[5-(isoxazol-3- | II_1 and 4- | rt: 4.973 |
| | ylaminomethyl)isoxazol-3-yl]phenyl}piperazin- | aminobenzoic acid | m/z: 520 |
| | 1-yl)-2-oxoethyl]benzamide | (IIIa_29) | |
| I_38 | 2-Amino-N-[2-(4-{2-fluoro-4-[5-(isoxazol-3- | II_1 and 2- | rt: 5.564 |
| | ylaminomethyl)isoxazol-3-yl]phenyl}piperazin- | aminobenzoic acid | m/z: 520 |
| | 1-yl)-2-oxoethyl]benzamide | (IIIa_30) | |
| I_39 | 3-Amino-N-[2-(4-{2-fluoro-4-[5-(isoxazol-3- | II_1 and 3- | rt: 4.778 |
| | ylaminomethyl)isoxazol-3-yl]phenyl}piperazin- | aminobenzoic acid | m/z: 520 |
| | 1-yl)-2-oxoethyl]benzamide | (IIIa_31) | |
| I_40 | 3,4-Diamino-N-[2-(4-{2-fluoro-4-[5-(isoxazol-3- | II_1 and 3,4- | rt: 4.384 |
| | ylaminomethyl)isoxazol-3-yl]phenyl}piperazin- | diaminobenzoic | m/z: 535 |
| | 1-yl)-2-oxoethyl]benzamide | acid (IIIa_32) | |
| I_41 | 3,5-Diamino-N-[2-(4-{2-fluoro-4-[5-(isoxazol-3- | II_1 and 3,5- | rt: 4.168 |
| | ylaminomethyl)isoxazol-3-yl]phenyl}piperazin- | diaminobenzoic | m/z: 535 |
| | 1-yl)-2-oxoethyl]benzamide | acid (IIIa_33) | |
| I_42 | 2-Amino-N-[2-(4-{2-fluoro-4-[5-(isoxazol-3- | II_1 and 2-amino-5- | rt: 5.824 |
| | ylaminomethyl)isoxazol-3-yl]phenyl}piperazin- | nitrobenzoic acid | m/z: 565 |
| | 1-yl)-2-oxoethyl]-5-nitrobenzamide | (IIIa_34) | |
| I_43 | 3-Dimethylamino-N-[2-(4-{2-fluoro-4-[5- | II_1 and 3- | tr: 5.574 |
| | (isoxazol-3-ylaminomethyl)isoxazol-3-yl]- | dimethylaminobenzoic | m/z: 548 |
| | phenyl}piperazin-1-yl)-2-oxoethyl]benzamide | acid (IIIa_35) | |
| I_44 | N-[2-(4-{2-Fluoro-4-[5-(isoxazol-3- | II_1 and 3,4- | rt: 5.708 |
| | ylaminomethyl)isoxazol-3-yl]phenyl}piperazin- | (methyl- | m/z: 549 |
| | 1-yl)-2-oxoethyl]benzo[1,3]dioxole-5- | endioxy)benzoyl | |
| | carboxamide | chloride (IIIb_8) | |
| I_45 | 3-Cyano-N-[2-(4-{2-fluoro-4-[5-(isoxazol-3- | II_1 and 3-cyano- | rt: 5.667 |
| | ylaminomethyl)isoxazol-3-yl]phenyl}piperazin- | benzoyl chloride | m/z: 530 |
| | 1-yl)-2-oxoethyl]benzamide | (IIIb_9) | |
| I_46 | 2-Cyano-N-[2-(4-{2-fluoro-4-[5-(isoxazol-3- | II_1 and 2- | rt: 5.254 |
| | ylaminomethyl)isoxazol-3-yl]phenyl}piperazin- | cyanobenzoic acid | m/z: 530 |
| | 1-yl)-2-oxoethyl]benzamide | (IIIa_36) | |
| I_47 | 2-Fluoro-N-[2-(4-{2-fluoro-4-[5-(isoxazol-3- | II_1 and 2- | rt: 6.004 |
| | ylaminomethyl)isoxazol-3-yl]phenyl}piperazin- | fluorobenzoic acid | m/z: 523 |
| | 1-yl)-2-oxoethyl]benzamide | (IIIa_37) | |
| I_48 | N-[2-(4-{2-Fluoro-4-[5-(isoxazol-3- | II_1 and 4- | rt: 4.283 |
| | ylaminomethyl)isoxazol-3-yl]phenyl}piperazin- | (imidazol-1- | m/z: 571 |
| | 1-yl)-2-oxoethyl]-4-(imidazol-1-yl)benzamide | yl)benzoic acid | |
| | | (IIIa_38) | |
| I_49 | N-[2-(4-{2-Fluoro-4-[5-(isoxazol-3- | II_1 and 3- | rt: 4.360 |
| | ylaminomethyl)isoxazol-3-yl]phenyl}piperazin- | (imidazol-1- | m/z: 571 |
| | 1-yl)-2-oxoethyl]-3-imidazol-1-ylbenzamide | yl)benzoic acid | |
| | | (IIIa_39) | |
| I_50 | N-[2-(4-{2-Fluoro-4-[5-isoxazol-3- | II_1 and 3-(pyrazol- | rt: 5.846 |
| | ylaminomethyl)isoxazol-3-yl]phenyl}piperazin- | 1-yl)benzoic acid | m/z: 571 |
| | 1-yl)-2-oxoethyl]-3-pyrazol-1-ylbenzamide | (IIIa_40) | |
| I_51 | N-[2-(4-{2-Fluoro-4-[5-(isoxazol-3- | II_1 and 3-(2- | rt: 6.264 |
| | ylaminomethyl)isoxazol-3-yl]phenyl}piperazin- | methylthiazol-5- | m/z: 602 |
| | 1-yl)-2-oxoethyl]-3-(2-methylthiazol-5- | yl)benzoic acid | |
| | yl)benzamide | (IIIa_41) | |
| I_52 | N-[2-(4-{2-Fluoro-4-[5-(isoxazol-3- | II_1 and 4-(pyridin- | rt: 4.548 |
| | ylaminomethyl)isoxazol-3-yl]phenyl}piperazin- | 4-yl)benzoic acid | m/z: 582 |
| | 1-yl)-2-oxoethyl]-4-pyridin-4-ylbenzamide | (IIIa_42) | |
| I_53 | N-[2-(4-{2-Fluoro-4-[5-(isoxazol-3- | II_1 and 3-(pyridin- | rt: 4.564 |
| | ylaminomethyl)isoxazol-3-yl]phenyl}piperazin- | 4-yl)benzoic acid | m/z: 582 |
| | 1-yl)-2-oxoethyl]-3-pyridin-4-ylbenzamide | (IIIa_43) | |
| I_54 | N-[2-(4-{2-Fluoro-4-[5-(isoxazol-3- | II_1 and 5- | rt: 5.954 |
| | ylaminomethyl)isoxazol-3-yl]phenyl}piperazin- | methylthiophene-2- | m/z: 525 |
| | 1-yl)-2-oxoethyl]-5-methylthiophene-2- | carboxylic acid | |
| | carboxamide | (IIIa_44) | |
| I_55 | 5-Bromo-N-[2-(4-{2-fluoro-4-[5-(isoxazol-3- | II_1 and 5- | rt: 6.352 |
| | ylaminomethyl)isoxazol-3-yl]phenyl}piperazin- | bromothiophene-2- | m/z: 589, 591 |
| | 1-yl)-2-oxoethyl]thiophene-2-carboxamide | carboxylic acid | |
| | | (IIIa_45) | |
| I_56 | 4,5-Dibromo-N-[2-(4-{2-fluoro-4-[5-(isoxazol-3- | II_1 and 4,5- | rt: 6.933 |
| | ylaminomethyl)isoxazol-3-yl]phenyl}piperazin- | dibromothiophene- | m/z: 667, 669, |
| | 1-yl)-2-oxoethyl]thiophene-2-carboxamide | 2-carboxylic acid | 671 |
| | | (IIIa_46) | |
| I_57 | 5-Chloro-N-[2-(4-{2-fluoro-4-[5-(isoxazol-3- | II_1 and 5- | rt: 6.293 |
| | ylaminomethyl)isoxazol-3-yl]phenyl}piperazin- | chlorothiophene-2- | m/z: 545, 547 |
| | 1-yl)-2-oxoethyl]thiophene-2-carboxamide | carboxylic acid | |
| | | (IIIa_47) | |
| I_58 | N-[2-(4-{2-Fluoro-4-[5-(isoxazol-3- | II_1 and 5- | rt: 5.663 |
| | ylaminomethyl)isoxazol-3-yl]phenyl}piperazin- | nitrofuran-2- | m/z: 540 |
| | 1-yl)-2-oxoethyl]-5-nitrofuran-2-carboxamide | carbonyl chloride | |
| | | (IIIb_10) | |
| I_59 | 5-Bromo-N-[2-(4-{2-fluoro-4-[5-(isoxazol-3- | II_1 and 5-bromo- | rt: 5.993 |
| | ylaminomethyl)isoxazol-3-yl]phenyl}piperazin- | furan-2-carboxylic | m/z: 573, 575 |
| | 1-yl)-2-oxoethyl]furan-2-carboxamide | acid (IIIa_48) | |
| I_60 | N-[2-(4-{2-Fluoro-4-[5-(isoxazol-3- | II_1 and furan-2- | rt: 5.352 |
| | ylaminomethyl)isoxazol-3-yl]phenyl}piperazin- | carbonyl chloride | m/z: 495 |
| | 1-yl)-2-oxoethyl]furan-2-carboxamide | (IIIb_11) | |
| I_61 | N-[2-(4-{2-Fluoro-4-[5-(isoxazol-3- | II_1 and furan-3- | rt: 5.279 |
| | ylaminomethyl)isoxazol-3-yl]phenyl}piperazin- | carboxylic acid | m/z: 495 |
| | 1-yl)-2-oxoethyl]furan-3-carboxamide | (IIIa_49) | |
| I_62 | N-[2-(4-{2-Fluoro-4-[5-(isoxazol-3- | II_1 and | rt: 6.339 |
| | ylaminomethyl)isoxazol-3-yl]phenyl}piperazin- | benzofuran-2- | m/z: 545 |
| | 1-yl)-2-oxoethyl]benzofuran-2-carboxamide | carboxylic acid | |
| | | (IIIa_50) | |
| I_63 | N-[2-(4-{2-Fluoro-4-[5-(isoxazol-3- | II_1 and pyrrole-2- | rt: 5.286 |
| | ylaminomethyl)isoxazol-3-yl]phenyl}piperazin- | carboxylic acid | m/z: 494 |
| | 1-yl)-2-oxoethyl]-1H-pyrrole-2-carboxamide | (IIIa_51) | |
| I_64 | N-[2-(4-{2-Fluoro-4-[5-(isoxazol-3- | II_1 and 1- | rt: 5.702 |
| | ylaminomethyl)isoxazol-3-yl]phenyl}piperazin- | methylpyrrole-2- | m/z: 508 |
| | 1-yl)-2-oxoethyl]-1-methyl-1H-pyrrole-2- | carboxylic acid | |
| | carboxamide | (IIIa_52) | |
| I_65 | N-[2-(4-{2-Fluoro-4-[5-(isoxazol-3- | II_1 and pyrrole-3- | rt: 4.891 |
| | ylaminomethyl)isoxazol-3-yl]phenyl}piperazin- | carboxylic acid | m/z: 494 |
| | 1-yl)-2-oxoethyl]-1H-pyrrole-3-carboxamide | (IIIa_53) | |
| I_66 | N-[2-(4-{2-Fluoro-4-[5-(isoxazol-3- | II_1 and imidazole- | rt: 4.238 |
| | ylaminomethyl)isoxazol-3-yl]phenyl}piperazin- | 4-carboxylic acid | m/z: 495 |
| | 1-yl)-2-oxoethyl]-1H-imidazole-4-carboxamide | (IIIa_54) | |
| I_67 | N-[2-(4-{2-Fluoro-4-[5-(isoxazol-3- | II_1 and pyrazole- | rt: 4.566 |
| | ylaminomethyl)isoxazol-3-yl]phenyl}piperazin- | 4-carboxylic acid | m/z: 495 |
| | 1-yl)-2-oxoethyl]-1H-pyrazole-4-carboxamide | (IIIa_55) | |
| I_68 | N-[2-(4-{2-Fluoro-4-[5-(isoxazol-3- | II_1 and 5- | rt: 5.308 |
| | ylaminomethyl)isoxazol-3-yl]phenyl}piperazin- | nitropyrazole-3- | m/z: 540 |
| | 1-yl)-2-oxoethyl]-5-nitro-1H-pyrazole-3- | carboxylic acid | |
| | carboxamide | (IIIa_56) | |
| I_69 | N-[2-(4-{2-Fluoro-4-[5-(isoxazol-3- | II_1 and 4- | rt: 5.141 |
| | ylaminomethyl)isoxazol-3-yl]phenyl}piperazin- | nitropyrazole-3- | m/z: 540 |
| | 1-yl)-2-oxoethyl]-4-nitro-1H-pyrazole-3- | carboxylic acid | |
| | carboxamide | (IIIa_57) | |
| I_70 | N-[2-(4-{2-Fluoro-4-[5-(isoxazol-3- | II_1 and 5- | rt: 5.005 |
| | ylaminomethyl)isoxazol-3-yl]phenyl}piperazin- | methylpyrazole-3- | m/z: 509 |
| | 1-yl)-2-oxoethyl]-5-methyl-1H-pyrazole-3- | carboxylic acid | |
| | carboxamide | (IIIa_58) | |
| I_71 | 3-Amino-N-[2-(4-{2-fluoro-4-[5-(isoxazol-3- | II_1 and 3- | rt: 4.496 |
| | ylaminomethyl)isoxazol-3-yl]phenyl}piperazin- | aminopyrazole-4- | m/z: 510 |
| | 1-yl)-2-oxoethyl]-1H-pyrazole-4-carboxamide | carboxylic acid | |
| | | (IIIa_59) | |
| I_72 | N-[2-(4-{2-Fluoro-4-[5-(isoxazol-3- | II_1 and indole-2- | rt: 6.158 |
| | ylaminomethyl)isoxazol-3-yl]phenyl}piperazin- | carboxylic acid | m/z: 544 |
| | 1-yl)-2-oxoethyl]-1H-indole-2-carboxamide | (IIIa_60) | |
| I_73 | 5-Fluoro-N-[2-(4-{2-fluoro-4-[5-(isoxazol-3- | II_1 and 5- | rt: 6.269 |
| | ylaminomethyl)isoxazol-3-yl]phenyl}piperazin- | fluoroindole-2- | m/z: 562 |
| | 1-yl)-2-oxoethyl]-1H-indole-2-carboxamide | carboxylic acid | |
| | | (IIIa_61) | |
| I_74 | 5-Benciloxi-N-[2-(4-{2-fluoro-4-[5-(isoxazol-3- | II_1 and 5- | rt: 7.091 |
| | ylaminomethyl)isoxazol-3-yl]phenyl}piperazin- | benciloxindole-2- | m/z: 650 |
| | 1-yl)-2-oxoethyl]-1H-indole-2-carboxamide | carboxylic acid | |
| | | (IIIa_62) | |
| I_75 | N-[2-(4-{2-Fluoro-4-[5-(isoxazol-3- | II_1 and 1- | rt: 6.615 |
| | ylaminomethyl)isoxazol-3-yl]phenyl}piperazin- | methylindole-2- | m/z: 558 |
| | 1-yl)-2-oxoethyl]-1-methyl-1H-indole-2- | carboxylic acid | |
| | carboxamide | (IIIa_63) | |
| I_76 | 2,3-Dihydro-N-[2-(4-{2-fluoro-4-[5-(isoxazol-3- | II_1 and indoline-2- | rt: 6.157 |
| | ylaminomethyl)isoxazol-3-yl]phenyl}piperazin- | carboxylic acid | m/z: 546 |
| | 1-yl)-2-oxoethyl]-1H-indole-2-carboxamide | (IIIa_64) | |
| I_77 | N-[2-(4-{2-Fluoro-4-[5-(isoxazol-3- | II_1 and indole-6- | rt: 5.743 |
| | ylaminomethyl)isoxazol-3-yl]phenyl}piperazin- | carboxylic acid | m/z: 544 |
| | 1-yl)-2-oxoethyl]-1H-indole-6-carboxamide | (IIIa_65) | |
| I_78 | N-[2-(4-{2-Fluoro-4-[5-(isoxazol-3- | II_1 and indole-5- | rt: 5.566 |
| | ylaminomethyl)isoxazol-3-yl]phenyl}piperazin- | carboxylic acid | m/z: 544 |
| | 1-yl)-2-oxoethyl]-1H-indole-5-carboxamide | (IIIa_66) | |
| I_79 | N-[2-(4-{2-Fluoro-4-[5-(isoxazol-3- | II_1 and | rt: 4.276 |
| | ylaminomethyl)isoxazol-3-yl]phenyl}piperazin- | benzimidazole-5- | m/z: 545 |
| | 1-yl)-2-oxoethyl]-1H-benzimidazole-5- | carboxylic acid | |
| | carbaxamide | (IIIa_67) | |
| I_80 | N-[2-(4-{2-Fluoro-4-[5-(isoxazol-3- | II_1 and 2-methyl- | rt: 5.139 |
| | ylaminomethyl)isoxazol-3-yl]phenyl}piperazin- | benzimidazole-5- | m/z: 559 |
| | 1-yl)-2-oxoethyl]-2-methyl-1H-benzimidazole-5- | carboxylic acid | |
| | carboxamide | (IIIa_68) | |
| I_81 | 1,2-Dimethyl-N-[2-(4-{2-fluoro-4-[5-(isoxazol-3- | II_1 and 1,2- | rt: 4.328 |
| | ylaminomethyl)isoxazol-3-yl]phenyl}piperazin- | dimethyl- | m/z: 573 |
| | 1-yl)-2-oxoethyl]-1H-benzimidazole-5- | benzimidazole-5- | |
| | carboxamide | carboxylic acid | |
| | | (IIIa_69) | |
| I_82 | 1-Cyclopropylmethyl-N-[2-(4-{2-fluoro-4-[5- | II_1 and 1- | rt: 4.728 |
| | (isoxazol-3-ylaminomethyl)isoxazol-3- | cyclopropylmethyl- | m/z: 613 |
| | yl]phenyl}piperazin-1-yl)-2-oxoethyl]-2-methyl- | 2-methyl- | |
| | 1H-benzimidazole-5-carboxamide | benzimidazole-5- | |
| | | carboxylic acid | |
| | | (IIIa_70) | |
| I_83 | N-[2-(4-{2-Fluoro-4-[5-(isoxazol-3- | II_1 and 2-methyl- | rt: 4.717 |
| | ylaminomethyl)isoxazol-3-yl]phenyl}piperazin- | 1- | (method B) |
| | 1-yl)-2-oxoethyl]-2-methyl-1-propyl-1H- | propylbenzimidazole- | m/z: 601 |
| | benzimidazole-5-carboxamide | 5-carboxylic acid | |
| | | (IIIa_71) | |
| I_84 | N-[2-(4-{2-Fluoro-4-[5-(isoxazol-3- | II_1 and 2-methyl- | rt: 5.522 |
| | ylaminomethyl)isoxazol-3-yl]phenyl}piperazin- | 1-(2- | (method B) |
| | 1-yl)-2-oxoethyl]-2-methyl-1-prop-2-inyl-1H- | propynyl)benzimidazole- | m/z: 597 |
| | benzimidazole-5-carboxamide | 5-carboxylic | |
| | | acid (IIIa_72) | |
| I_85 | 1-Allyl-N-[2-(4-{2-fluoro-4-[5-(isoxazol-3- | II_1 and 1-allyl-2- | rt: 4.773 |
| | ylaminomethyl)isoxazol-3-yl]phenyl}piperazin- | methylbenzimidazole- | (method B) |
| | 1-yl)-2-oxoethyl]-2-methyl-1H-benzimidazole- | 5-carboxylic acid | m/z: 599 |
| | 5-carboxamide | (IIIa_73) | |
| I_86 | 1-Cyclopentyl-N-[2-(4-{2-fluoro-4-[5-(isoxazol- | II_1 and 1- | rt: 5.010 |
| | 3-ylaminomethyl)isoxazol-3- | cyclopentyl-2- | (method B) |
| | yl]phenyl}piperazin-1-yl)-2-oxoethyl]-2-methyl- | methylbenzimidazole- | m/z: 627 |
| | 1H-benzimidazole-5-carboxamide | 5-carboxylic | |
| | | acid (IIIa_74) | |
| I_87 | 1-Cyclohexyl-N-[2-(4-{2-fluoro-4-[5-(isoxazol-3- | II_1 and 1- | rt: 5.221 |
| | ylaminomethyl)isoxazol-3-yl]phenyl}piperazin- | cyclohexyl-2- | m/z: 641 |
| | 1-yl)-2-oxoethyl]-2-methyl-1H-benzimidazole-5- | methylbenzimidazole- | |
| | carboxamide | 5-carboxylic | |
| | | acid (IIIa_75) | |
| I_88 | N-[2-(4-{2-Fluoro-4-[5-(isoxazol-3- | II_1 and imidazo- | rt: 4.525 |
| | ylaminomethyl)isoxazol-3-yl]phenyl}piperazin- | [1,2-a]pyridine-3- | m/z: 545 |
| | 1-yl)-2-oxoethyl]imidazo[1,2-a]pyridine-3- | carboxylic acid | |
| | carboxamide | (IIIa_76) | |
| I_89 | N-[2-(4-{2-Fluoro-4-[5-(isoxazol-3- | II_1 and | rt: 4.953 |
| | ylaminomethyl)isoxazol-3-yl]phenyl}piperazin- | imidazo[1,2- | m/z: 545 |
| | 1-yl)-2-oxoethyl]imidazo[1,2-a]pyridine-2- | a]pyridine-2- | |
| | carboxamide | carboxylic acid | |
| | | (IIIa_77) | |
| I_90 | N-[2-(4-{2-Fluoro-4-[5-(isoxazol-3- | II_1 and indazole- | rt: 5.804 |
| | ylaminomethyl)isoxazol-3-yl]phenyl}piperazin- | 3-carboxylic acid | m/z: 545 |
| | 1-yl)-2-oxoethyl]-1H-indazole-3-carboxamide | (IIIa_78) | |
| I_91 | N-[2-(4-{2-Fluoro-4-[5-(isoxazol-3- | II_1 and isoxazole- | rt: 5.136 |
| | ylaminomethyl)isoxazol-3-yl]phenyl}piperazin- | 5-carbonyl chloride | m/z: 496 |
| | 1-yl)-2-oxoethyl]isoxazole-5-carboxamide | (IIIb_12) | |
| I_92 | N-[2-(4-{2-Fluoro-4-[5-(isoxazol-3- | II_1 and 5- | rt: 5.634 |
| | ylaminomethyl)isoxazol-3-yl]phenyl}piperazin- | methylisoxazole-3- | m/z: 510 |
| | 1-yl)-2-oxoethyl]-5-methylisoxazole-3- | carboxylic acid | |
| | carboxamide | (IIIa_79) | |
| I_93 | N-[2-(4-{2-Fluoro-4-[5-(isoxazol-3- | II_1 and pyridine-3- | rt: 4.632 |
| | ylaminomethyl)isoxazol-3-yl]phenyl}piperazin- | carbonyl chloride | m/z: 506 |
| | 1-yl)-2-oxoethyl]nicotinamide | (IIIb_13) | |
| I_94 | N-[2-(4-{2-Fluoro-4-[5-(isoxazol-3- | II_1 and pyridine-4- | rt: 4.561 |
| | ylaminomethyl)isoxazol-3-yl]phenyl}piperazin- | carboxylic acid | m/z: 506 |
| | 1-yl)-2-oxoethyl]isonicotinamide | (IIIa_80) | |
| I_95 | N-[2-(4-{2-Fluoro-4-[5-(isoxazol-3- | II_1 and pyridine-2- | rt: 5.638 |
| | ylaminomethyl)isoxazol-3-yl]phenyl}piperazin- | carboxylic acid | m/z: 506 |
| | 1-yl)-2-oxoethyl]pyridine-2-carboxamide | (IIIa_81) | |
| I_96 | 2-Chloro-N-[2-(4-{2-fluoro-4-[5-(isoxazol-3- | II_1 and 2- | rt: 5.256 |
| | ylaminomethyl)isoxazol-3-yl]phenyl}piperazin- | chloropyridine-3- | m/z: 540, 542 |
| | 1-yl)-2-oxoethyl]nicotinamide | carboxylic acid | |
| | | (IIIa_82) | |
| I_97 | 6-Chloro-N-[2-(4-{2-fluoro-4-[5-(isoxazol-3- | II_1 and 6- | rt: 5.544 |
| | ylaminomethyl)isoxazol-3-yl]phenyl}piperazin- | chloropyridine-3- | m/z: 540, 542 |
| | 1-yl)-2-oxoethyl]nicotinamide | carboxylic acid | |
| | | (IIIa_83) | |
| I_98 | 2,6-Dichloro-N-[2-(4-{2-fluoro-4-[5-(isoxazol-3- | II_1 and 2,6- | rt: 5.968 |
| | ylaminomethyl)isoxazol-3-yl]phenyl}piperazin- | dichloropyridine-3- | m/z: 573, 575, |
| | 1-yl)-2-oxoethyl]nicotinamide | carboxylic acid | 576, 577 |
| | | (IIIa_84) | |
| I_99 | 5-Bromo-N-[2-(4-{2-fluoro-4-[5-isoxazol-3- | II_1 and 5- | rt: 5.623 |
| | ylaminomethyl)isoxazol-3-yl]phenyl}-piperazin- | bromopyridine-3- | m/z: 583, 585 |
| | 1-yl)-2-oxoethyl]nicotinamide | carboxylic acid | |
| | | (IIIa_85) | |
| I_100 | N-[2-(4-{2-Fluoro-4-[5-(isoxazol-3- | II_1 and 6- | rt: 5.687 |
| | ylaminomethyl)isoxazol-3-yl]phenyl}piperazin- | methylpyridine-3- | m/z: 520 |
| | 1-yl)-2-oxoethyl]-6-methylnicotinamide | carboxylic acid | |
| | | (IIIa_86) | |
| I_101 | N-[2-(4-{2-Fluoro-4-[5-(isoxazol-3- | II_1 and pyridine-3- | rt: 4.424 |
| | ylaminomethyl)isoxazol-3-yl]phenyl}piperazin- | carboxylic acid N- | m/z: 522 |
| | 1-yl)-2-oxoethyl]nicotinamide 1-oxide | oxide (IIIa_87) | |
| I_102 | N-[2-(4-{2-Fluoro-4-[5-(isoxazol-3- | II_1 and pyridine-2- | rt: 4.913 |
| | ylaminomethyl)isoxazol-3-yl]phenyl}piperazin- | carboxylic acid N- | m/z: 522 |
| | 1-yl)-2-oxoethyl]pyridine-2-carboxamide 1- | oxide (IIIa_88) | |
| | oxide | | |
| I_103 | N-[2-(4-{2-Fluoro-4-[5-(isoxazol-3- | II_1 and pyridine-4- | rt: 4.426 |
| | ylaminomethyl)isoxazol-3-yl]phenyl}piperazin- | carboxylic acid N- | m/z: 522 |
| | 1-yl)-2-oxoethyl]isonicotinamide 1-oxide | oxide (IIIa_89) | |
| I_104 | N-[2-(4-{2-Fluoro-4-[5-(isoxazol-3- | II_1 and 4-nitropyridine- | rt: 4.911 |
| | ylaminomethyl)isoxazol-3-yl]phenyl}piperazin- | 3-carboxylic | m/z: 567 |
| | 1-yl)-2-oxoethyl]-4-nitronicotinamide 1-oxide | acid N-oxide | |
| | | (IIIa_90) | |
| I_105 | N-[2-(4-{2-Fluoro-4-[5-(isoxazol-3- | II_1 and 6-hydroxy- | rt: 4.510 |
| | ylaminomethyl)isoxazol-3-yl]phenyl}piperazin- | pyridine-3- | m/z: 522 |
| | 1-yl)-2-oxoethyl]-6-hydroxynicotinamide | carboxylic acid | |
| | | (IIIa_91) | |
| I_106 | N-[2-(4-{2-Fluoro-4-[5-(isoxazol-3- | II_1 and 6- | rt: 4.732 |
| | ylaminomethyl)isoxazol-3-yl]phenyl}piperazin- | hydroxypyridine-2- | m/z: 522 |
| | 1-yl)-2-oxoethyl]-6-hydroxypyridine-2- | carboxylic acid | |
| | carboxamide | (IIIa_92) | |
| I_107 | N-[2-(4-{2-Fluoro-4-[5-(isoxazol-3- | II_1 and 3- | rt: 6.218 |
| | ylaminomethyl)isoxazol-3-yl]phenyl}piperazin- | hydroxypyridine-2- | m/z: 522 |
| | 1-yl)-2-oxoethyl]-3-hydroxypyridine-2- | carboxylic acid | |
| | carboxamide | (IIIa_93) | |
| I_108 | N-[2-(4-{2-Fluoro-4-[5-(isoxazol-3- | II_1 and 2- | rt: 5.787 |
| | ylaminomethyl)isoxazol-3-yl]phenyl}piperazin- | methoxypyridine-3- | m/z: 536 |
| | 1-yl)-2-oxoethyl]-2-methoxynicotinamide | carboxylic acid | |
| | | (IIIa_94) | |
| I_109 | 2-Ethoxy-N-[2-(4-{2-fluoro-4-[5-(isoxazol-3- | II_1 and 2-ethoxy- | rt: 6.340 |
| | ylaminomethyl)isoxazol-3-yl]phenyl}piperazin- | pyridine-3- | m/z: 550 |
| | 1-yl)-2-oxoethyl]nicotinamide | carboxylic acid | |
| | | (IIIa_95) | |
| I_110 | N-[2-(4-{2-Fluoro-4-[5-(isoxazol-3- | II_1 and 4- | rt: 5.733 |
| | ylaminomethyl)isoxazol-3-yl]phenyl}piperazin- | methoxy-pyridine- | m/z: 536 |
| | 1-yl)-2-oxoethyl]-4-methoxypyridine-2- | 2-carboxylic acid | |
| | carboxamide | (IIIa_96) | |
| I_111 | 2-Amino-N-[2-(4-{2-fluoro-4-[5-(isoxazol-3- | II_1 and 2-amino- | rt: 4.212 |
| | ylaminomethyl)isoxazol-3-yl]phenyl}piperazin- | pyridine-3- | m/z: 521 |
| | 1-yl)-2-oxoethyl]nicotinamide | carboxylic acid | |
| | | (IIIa_97) | |
| I_112 | 6-Amino-N-[2-(4-{2-fluoro-4-[5-(isoxazol-3- | II_1 and 6-amino- | rt: 4.081 |
| | ylaminomethyl)isoxazol-3-yl]phenyl}piperazin- | pyridine-3- | m/z: 521 |
| | 1-yl)-2-oxoethyl]nicotinamide | carboxylic acid | |
| | | (IIIa_83) | |
| I_113 | 6-Amino-N-[2-(4-{2-fluoro-4-[5-(isoxazol-3- | II_1 and 6-amino- | rt: 6.697 |
| | ylaminomethyl)isoxazol-3-yl]phenyl}piperazin- | pyridine-2- | m/z 521 |
| | 1-yl)-2-oxoethyl]pyridine-2-carboxamide | carboxylic acid | |
| | | (IIIa_98) | |
| I_114 | 2-Amino-N-[2-(4-{2-fluoro-4-[5-(isoxazol-3- | II_1 and 2-amino- | rt: 4.116 |
| | ylaminomethyl)isoxazol-3-yl]phenyl}piperazin- | pyridine-4- | m/z: 521 |
| | 1-yl)-2-oxoethyl]isonicotinamide | carboxylic acid | |
| | | (IIIa_99) | |
| I_115 | 2-Amino-4,6-dimethyl-N-[2-(4-({2-fluoro-4-[5- | II_1 and 2-amino- | rt: 4.313 |
| | (isoxazol-3-ylaminomethyl)isoxazol-3- | 4,6- | m/z: 549 |
| | yl]phenyl}piperazin-1-yl)-2- | dimethylpyridin-3- | |
| | oxoethyl]nicotinamide | carboxylic acid | |
| | | (IIIa_100) | |
| I_116 | 4-(2,2-Dimethylpropionylamino)-N-[2-(4-{2- | II_1 and 4-(2,2- | rt: 5.600 |
| | fluoro-4-[5-(isoxazol-3-ylaminomethyl)isoxazol- | dimethylpropionyl- | m/z: 605 |
| | 3-yl]phenyl}piperazin-1-yl)-2- | amino)pyridine-3- | |
| | oxoethyl]nicotinamide | carboxylic acid | |
| | | (IIIa_101) | |
| I_117 | 6-Acetylamino-N-[2-(4-{2-fluoro-4-[5-(isoxazol- | II_1 and 6- | rt: 4.917 |
| | 3-ylaminomethyl)isoxazol-3- | acetylaminopyridine- | m/z: 563 |
| | yl]phenyl}piperazin-1-yl)-2- | 3-carboxylic acid | |
| | oxoethyl]nicotinamide | (IIIa_102) | |
| I_118 | 6-[2-(4-{2-Fluoro-4-[5-(isoxazol-3- | II_1 and pyridin- | rt: 5.209 |
| | ylaminomethyl)isoxazol-3-yl]phenyl}piperazin- | 2,5-dicarboxylic | m/z: 550 |
| | 1-yl)-2-oxoethylcarbamoyl]nicotinic acid | acid (IIIa_103) | |
| I_119 | N-[2-(4-{2-Fluoro-4-[5-(isoxazol-3- | II_1 and 6- | rt: 5.998 |
| | ylaminomethyl)isoxazol-3-yl]phenyl}piperazin- | trifluoromethyl- | m/z: 574 |
| | 1-yl)-2-oxoethyl]-6-trifluoromethylnicotinamide | pyridine-3- | |
| | | carboxylic acid | |
| | | (IIIa_104) | |
| I_120 | N-[2-(4-{2-Fluoro-4-[5-(isoxazol-3- | II_1 and | rt: 6.375 |
| | ylaminomethyl)isoxazol-3-yl]phenyl}piperazin- | isoquinoline-1- | m/z: 556 |
| | 1-yl)-2-oxoethyl]isoquinoline-1-carboxamide | carboxylic acid | |
| | | (IIIa_105) | |
| I_121 | N-[2-(4-{2-Fluoro-4-[5-(isoxazol-3- | II_1 and quinoline- | rt: 6.567 |
| | ylaminomethyl)isoxazol-3-yl]phenyl}piperazin- | 2-carboxylic acid | m/z: 556 |
| | 1-yl)-2-oxoethyl]quinoline-2-carboxamide | (IIIa_106) | |
| I_122 | N-[2-(4-{2-Fluoro-4-[5-(isoxazol-3- | II_1 and quinoline- | rt: 5.962 |
| | ylaminomethyl)isoxazol-3-yl]phenyl}piperazin- | 8-carboxylic acid | m/z: 556 |
| | 1-yl)-2-oxoethyl]quinoline-8-carboxamide | (IIIa_107) | |
| I_123 | N-[2-(4-{2-fluoro-4-[5-(isoxazol-3- | II_1 and quinoline- | rt: 5.455 |
| | ylaminomethyl)isoxazol-3-yl]phenyl}piperazin- | 3-carboxylic acid | m/z: 556 |
| | 1-yl)-2-oxoethyl]quinoline-3-carboxamide | (IIIa_108) | |
| I_124 | N-[2-(4-{2-Fluoro-4-[5-(isoxazol-3- | II_1 and quinoline- | rt: 5.202 |
| | ylaminomethyl)isoxazol-3-yl]phenyl}piperazin- | 4-carboxylic acid | m/z: 556 |
| | 1-yl)-2-oxoethyl]quinoline-4-carboxamide | (IIIa_109) | |
| I_125 | N-[2-(4-{2-Fluoro-4-[5-(isoxazol-3- | II_1 and quinoline- | rt: 4.808 |
| | ylaminomethyl)isoxazol-3-yl]phenyl}piperazin- | 5-carboxylic acid | m/z: 556 |
| | 1-yl)-2-oxoethyl]quinoline-5-carboxamide | (IIIa_110) | |
| I_126 | N-[2-(4-{2-Fluoro-4-[5-(isoxazol-3- | II_1 and 2- | rt: 5.116 |
| | ylaminomethyl)isoxazol-3-yl]phenyl}piperazin- | hydroxyquinoline-4- | m/z: 572 |
| | 1-yl)-2-oxoethyl]-2-hydroxyquinoline-4- | carboxylic acid | |
| | carboxamide | (IIIa_111) | |
| I_127 | N-[2-(4-{2-Fluoro-4-[5-(isoxazol-3- | II_1 and quinoline- | rt: 4.909 |
| | ylaminomethyl)isoxazol-3-yl]phenyl}piperazin- | 6-carboxylic acid | m/z: 556 |
| | 1-yl)-2-oxoethyl]quinoline-6-carboxamide | (IIIa_112) | |
| I_128 | N-[2-(4-{2-Fluoro-4-[5-(isoxazol-3- | II_1 and 8- | rt: 6.212 |
| | ylaminomethyl)isoxazol-3-yl]phenyl}piperazin- | hydroxyquinoline-2- | m/z: 572 |
| | 1-yl)-2-oxoethyl]-8-hydroxyquinoline-2- | carboxylic acid | |
| | carboxamide | (IIIa_113) | |
| I_129 | N-[2-(4-{2-Fluoro-4-[5-(isoxazol-3- | II_1 and | rt: 6.103 |
| | ylaminomethyl)isoxazol-3-yl]phenyl}piperazin- | quinoxaline-2- | m/z: 557 |
| | 1-yl)-2-oxoethyl]quinoxaline-2-carboxamide | carboxylic acid | |
| | | (IIIa_114) | |
| I_130 | N-[2-(4-{2-Fluoro-4-[5-(isoxazol-3- | II_1 and 3- | rt: 5.098 |
| | ylaminomethyl)isoxazol-3-yl]phenyl}piperazin- | hydroxyquinoxaline- | m/z: 573 |
| | 1-yl)-2-oxoethyl]-3-hydroxyquinoxaline-2- | 2-carboxylic acid | |
| | carboxamide | (IIIa_115) | |
| I_131 | 2,4-Dihydroxy-N-[2-(4-{2-fluoro-4-[5-(isoxazol- | II_1 and 2,4- | rt: 4.669 |
| | 3-ylaminomethyl)isoxazol-3- | dihydroxypyrimidine- | m/z: 539 |
| | yl]phenyl}piperazin-1-yl)-2-oxoethyl]pyrimidine- | 5-carboxylic acid | |
| | 5-carboxamide | (IIIa_116) | |
| I_132 | N-[2-(4-{2-Fluoro-4-[5-(isoxazol-3- | II_1 and 3- | rt: 4.915 |
| | ylaminomethyl)isoxazol-3-yl]phenyl}piperazin- | hydroxysoxazole-5- | m/z: 512 |
| | 1-yl)-2-oxoethyl]-3-hydroxysoxazole-5- | carboxylic acid | |
| | carboxamide | (IIIa_117) | |
| I_133 | N-[2-(4-{2-Fluoro-4-[5-(isoxazol-3- | II_1 and 2- | rt: 4.751 |
| | ylaminomethyl)isoxazol-3-yl]phenyl}piperazin- | hydroxypyridine-3- | m/z: 522 |
| | 1-yl)-2-oxoethyl]-2-hydroxynicotinamide | carboxylic acid | |
| | | (IIIa_118) | |
| I_134 | 3-Amino-N-[2-(4-{2-fluoro-4-[5-(isoxazol-3- | II_1 and 3-amino- | rt: 5.374 |
| | ylaminomethyl)isoxazol-3-yl]phenyl}piperazin- | pyridine-2- | m/z: 521 |
| | 1-yl)-2-oxoethyl]pyridine-2-carboxamide | carboxylic acid | |
| | | (IIIa_119) | |
| I_135 | 3-Amino-N-[2-(4-{2-fluoro-4-[5-(isoxazol-3- | II_1 and 3-amino- | rt: 4.189 |
| | ylaminomethyl)isoxazol-3-yl]phenyl}piperazin- | pyridine-4- | m/z: 521 |
| | 1-yl)-2-oxoethyl]isonicotinamide | carboxylic acid | |
| | | (IIIa_120) | |
| I_136 | N-[2-(4-{2-Fluoro-4-[5-(isoxazol-3- | II_1 and | rt: 4.434 |
| | ylaminomethyl)isoxazol-3-yl]phenyl}piperazin- | isoquinoline-5- | m/z: 556 |
| | 1-yl)-2-oxoethyl]isoquinoline-5-carboxamide | carboxylic acid | |
| | | (IIIa_121) | |
| I_137 | N-[2-(4-{2-Fluoro-4-[5-(isoxazol-3- | II_1 and | rt: 5.207 |
| | ylaminomethyl)isoxazol-3-yl]phenyl}piperazin- | quinoxaline-6- | m/z: 557 |
| | 1-yl)-2-oxoethyl]quinoxaline-6-carboxamide | carboxylic acid | |
| | | (IIIa_122) | |
| I_138 | 4-Amino-N-[2-(4-{2-fluoro-4-[5-(isoxazol-3- | II_1 and 4-amino- | rt: 4.119 |
| | ylaminomethyl)isoxazol-3-yl]phenyl}piperazin- | pyridine-3- | m/z: 521 |
| | 1-yl)-2-oxoethyl]nicotinamide | carboxylic acid | |
| | | (IIIa_123) | |
| I_139 | 5-Amino-N-[2-(4-{2-fluoro-4-[5-(isoxazol-3- | II_1 and 5-amino- | rt: 4.140 |
| | ylaminomethyl)isoxazol-3-yl]phenyl}piperazin- | pyridine-3- | m/z: 521 |
| | 1-yl)-2-oxoethyl]nicotinamide | carboxylic acid | |
| | | (IIIa_124) | |
| I_140 | N-[2-(4-{2-Fluoro-4-[5-(isoxazol-3- | II_1 and 3- | rt: 4.065 |
| | ylaminomethyl)isoxazol-3-yl]phenyl}piperazin- | pyridylacetic acid | m/z: 520 |
| | 1-yl)-2-oxoethyl]-2-pyridin-3-ylacetamide | (IIIa_125) | |
| I_141 | N-[2-(4-{2-Fluoro-4-[5-(isoxazol-3- | II_1 and 2- | rt: 4.228 |
| | ylaminomethyl)isoxazol-3-yl]phenyl}piperazin- | pyridylacetic acid | m/z: 520 |
| | 1-yl)-2-oxoethyl]-2-pyridin-2-ylacetamide | (IIIa_126) | |
| I_142 | N-[2-(4-{2-Fluoro-4-[5-(isoxazol-3- | II_1 and 4-imidazolylacetic | rt: 3.962 |
| | ylaminomethyl)isoxazol-3-yl]phenyl}piperazin- | acid(IIIa_127) | m/z: 509 |
| | 1-yl)-2-oxoethyl]-2-(1H-imidazol-4-yl)acetamide | | |
| I_143 | N-[2-(4-{2-Fluoro-4-[5-(isoxazol-3- | II_1 and (1-methyl- | rt: 6.194 |
| | ylaminomethyl)isoxazol-3-yl]phenyl}piperazin- | indol-4-yl)acetic | m/z: 572 |
| | 1-yl)-2-oxoethyl]-2-(1-methyl-1H-indol-3- | acid(IIIa_128) | |
| | yl)acetamide | | |
| I_144 | N-[2-(4-{2-Fluoro-4-[5-(isoxazol-3- | II_1 and 4- | rt: 4.090 |
| | ylaminomethyl)isoxazol-3-yl]phenyl}piperazin- | imidazolylacrylic | m/z: 521 |
| | 1-yl)-2-oxoethyl]-3-(1H-imidazol-4- | acid (IIIa_129) | |
| | yl)acrylamide | | |
| I_145 | N-{[2-(4-{2-Fluoro-4-[5-(isoxazol-3- | II_1 and N-(2- | rt: 4.906 |
| | ylaminomethyl)isoxazol-3-yl]phenyl}piperazin- | furoyl)glycine | m/z: 552 |
| | 1-yl)-2-oxoethylcarbamoyl]methyl}furan-2- | (IIIa_130) | |
| | carboxamide | | |
| I_146 | 6-Amino-N-[2-(4-{2-fluoro-4-[5-(isoxazol-3- | II_2 and 6-amino- | rt: 4.302 |
| | ylaminomethyl)isoxazol-3-yl]phenyl}piperazin- | pyridine-3- | m/z: 535 |
| | 1-yl)-1-methyl-2-oxoethyl]nicotinamide | carboxylic acid | |
| | | (IIIa_83) | |
| I_147 | 6-Amino-N-[2-(4-{2-fluoro-4-[5-(isoxazol-3- | II_3 and 6-amino- | rt: 5.075 |
| | ylaminomethyl)isoxazol-3-yl]phenyl}piperazin- | pyridine-3- | m/z: 597 |
| | 1-yl)-2-oxo-1-phenylethyl]nicotinamide | carboxylic acid | |
| | | (IIIa_83) | |
| I_148 | N-[2-(4-{2-Fluoro-4-[5-(isoxazol-3- | II_3 and 6-methyl- | rt: 5.722 |
| | ylaminomethyl)isoxazol-3-yl]phenyl}piperazin- | pyridine-3- | m/z: 596 |
| | 1-yl)-2-oxo-1-phenylethyl]-6- | carboxylic acid | |
| | methylnicotinamide | (IIIa_86) | |
| I_149 | 2-Amino-N-[2-(4-{2-fluoro-4-[5-(isoxazol-3- | I_8 | rt: 3.825 |
| | ylaminomethyl)isoxazol-3-yl]phenyl}piperazin- | | m/z: 458 |
| | 1-yl)-2-oxoethyl]acetamide | | |
| I_150 | N-[2-(4-{4-[5-(Acetylaminomethyl)isoxazol-3- | II_4 and benzimidazole- | rt: 3.746 |
| | yl]-2-fluorophenyl}piperazin-1-yl)-2-oxoethyl]- | 5-carboxylic | m/z: 520 |
| | 1H-benzimidazole-5-carboxamide | acid (IIIa_67) | |
| I_151 | N-[2-(4-{2-Fluoro-4-[5-(isoxazol-3- | II_1 and 3-(1H- | rt: 4.017 |
| | ylaminomethyl)isoxazol-3-yl]phenyl}piperazin- | imidazol-4- | m/z: 523 |
| | 1-yl)-2-oxoethyl]-3-(1H-imidazol-4- | yl)propanoic acid | |
| | yl)propionamide | (IIIa_131) | |
| I_152 | N-[2-(4-{2-Fluoro-4-[5-(isoxazol-3- | II_1 and N-(9- | rt: 3.932 |
| | ylaminomethyl)isoxazol-3-yl]phenyl}piperazin- | fluorenylmethoxy- | m/z: 472 |
| | 1-yl)-2-oxoethyl]-2-methylaminoacetamide | carbonyl)-N-methyl- | |
| | | glycine (IIIa_132) | |
| I_153 | N-[2-(4-{2-Fluoro-4-[5-(isoxazol-3- | II_3 and | rt: 5.206 |
| | ylaminomethyl)isoxazol-3-yl]phenyl}piperazin- | benzimidazole-5- | m/z: 621 |
| | 1-yl)-2-oxo-1-phenylethyl]-1H-benzimidazole-5- | carboxylic acid | |
| | carboxamide | (IIIa_67) | |
| I_154 | 6-Amino-N-[2-(4-{2-fluoro-4-[5-(isoxazol-3- | II_5 and 6-amino- | rt: 4.137 |
| | ylaminomethyl)isoxazol-3-yl]phenyl}piperazin- | pyridine-3- | m/z: 535 |
| | 1-yl)-2-oxoethyl]-N-methylnicotinamide | carboxylic acid | |
| | | (IIIa_83) | |
| I_155 | N-[2-(4-{2-Fluoro-4-[5-(isoxazol-3- | II_5 and | rt: 4.265 |
| | ylaminomethyl)isoxazol-3-yl]phenyl}piperazin- | benzimidazole-5- | m/z: 559 |
| | 1-yl)-2-oxoethyl]-N-methyl-1H-benzimidazole- | carboxylic acid | |
| | 5-carboxamide | (IIIa_67) | |
| I_156 | N-[2-(4-{2-Fluoro-4-[5-(isoxazol-3- | II_5 and 6- | rt: 4.501 |
| | ylaminomethyl)isoxazol-3-yl]phenyl}piperazin- | methylpyridine-3- | m/z: 534 |
| | 1-yl)-2-oxoethyl]-6,N-dimethylnicotinamide | carboxylic acid | |
| | | (IIIa_86) | |
| I_157 | 6-Amino-N-[2-(4-{2,6-difluoro-4-[5-(isoxazol-3- | II_6 and 6-amino- | rt: 4.361 |
| | ylaminomethyl)isoxazol-3-yl]phenyl}piperazin- | pyridine-3- | m/z: 539 |
| | 1-yl)-2-oxoethyl]nicotinamide | carboxylic acid | |
| | | (IIIa_83) | |
| I_158 | N-[2-(4-{2,6-Difluoro-4-[5-(isoxazol-3- | II_6 and 6-methyl- | rt: 4.818 |
| | ylaminomethyl)isoxazol-3-yl]phenyl}piperazin- | pyridine-3- | m/z: 538 |
| | 1-yl)-2-oxoethyl]-6-methylnicotinamide | carboxylic acid | |
| | | (IIIa_86) | |
| I_159 | N-[2-(4-{2,6-Difluoro-4-[5-(isoxazol-3- | II_6 and benzimidazole- | rt: 4.466 |
| | ylaminomethyl)isoxazol-3-yl]phenyl}piperazin- | 5-carboxylic | m/z: 563 |
| | 1-yl)-2-oxoethyl]-1H-benzimidazole-5- | acid (IIIa_67) | |
| | carboxamide | | |
| I_160 | N-[2-(4-{2,6-Difluoro-4-[5-(isoxazol-3- | II_6 and 8-hydroxy- | rt: 6.533 |
| | ylaminomethyl)isoxazol-3-yl]phenyl}piperazin- | quinoline-2- | m/z: 590 |
| | 1-yl)-2-oxoethyl]-8-hydroxyquinoline-2- | carboxylic acid | |
| | carboxamide | (IIIa_113) | |
| I_161 | (S)-6-Amino-N-[2-(4-{2-fluoro-4-[5-(isoxazol-3- | II_7 and 6-amino- | rt: 4.307 |
| | ylaminomethyl)isoxazol-3-yl]phenyl}piperazin- | pyridine-3- | m/z: 535 |
| | 1-yl)-1-methyl-2-oxoethyl]nicotinamide | carboxylic acid | |
| | | (IIIa_083) | |
| I_162 | (R)-6-Amino-N-[2-(4-{2-fluoro-4-[5-(isoxazol-3- | II_8 and 6-amino- | rt: 4.310 |
| | ylaminomethyl)isoxazol-3-yl]phenyl}piperazin- | pyridine-3- | m/z: 535 |
| | 1-yl)-1-methyl-2-oxoethyl]nicotinamide | carboxylic acid | |
| | | (IIIa_083) | |
| I_163 | (R)—N-[2-(4-{2-Fluoro-4-[5-(isoxazol-3- | II_8 and 6-methyl- | rt: 4.307 |
| | ylaminomethyl)isoxazol-3-yl]phenyl}piperazin- | pyridine-3- | m/z: 534 |
| | 1-yl)-1-methyl-2-oxoethyl]-6- | carboxylic acid | |
| | methylnicotinamide | (IIIa_086) | |
| I_164 | (R)-3-Amino-N-[2-(4-{2-fluoro-4-[5-(isoxazol-3- | II_8 and 3-amino- | rt: 4.310 |
| | ylaminomethyl)isoxazol-3-yl]phenyl}piperazin- | pyridine-4- | m/z: 535 |
| | 1-yl)-1-methyl-2-oxoethyl]isonicotinamide | carboxylic acid | |
| | | (IIIa_120) | |
| I_165 | (R)—N-[2-(4-{2-Fluoro-4-[5-(isoxazol-3- | II_8 and pyrazole- | rt: 4.677 |
| | ylaminomethyl)isoxazol-3-yl]phenyl}piperazin- | 4-carboxylic acid | m/z: 509 |
| | 1-yl)-1-methyl-2-oxoethyl]pyrazole-4- | (IIIa_055) | |
| | carboxamide | | |
| I_166 | (R)—N-[1-(4-{2-Fluoro-4-[5-(isoxazol-3- | II_9 and | rt: 4.428 |
| | ylaminomethyl)isoxazol-3-yl]phenyl}piperazin- | benzimidazole-5- | m/z: 587 |
| | 1-carbonyl)-2-methylpropyl]-1H-benzimidazole- | carboxylic acid | |
| | 5-carboxamide | (IIIa_067) | |
| I_167 | (S)—N-[2-(4-{2-Fluoro-4-[5-(isoxazol-3- | II_7 and 6- | rt: 4.815 |
| | ylaminomethyl)isoxazol-3-yl]phenyl}piperazin- | methylpyridine-3- | m/z: 562 |
| | 1-yl)-1-methyl-2-oxoethyl]-6- | carboxylic acid | |
| | methylnicotinamide | (IIIa_086) | |
| I_168 | (S)-3-Amino-N-[1-(4-{2-fluoro-4-[5-(isoxazol-3- | II_10 and 3- | rt: 4.981 |
| | ylaminomethyl)isoxazol-3-yl]phenyl}piperazin- | aminopyridine-2- | m/z: 563 |
| | 1-carbonyl)-2-methylpropyl]pyridine-2- | carboxylic acid | |
| | carboxamide | (IIIa_119) | |
| I_169 | (R)—N-[1-(4-{2-Fluoro-4-[5-(isoxazol-3- | II_9 and 6- | rt: 5.355 |
| | ylaminomethyl)isoxazol-3-yl]phenyl}piperazin- | methylpyridine-3- | m/z: 534 |
| | 1-carbonyl)-2-methylpropyl]-6- | carboxylic acid | |
| | methylnicotinamide | (IIIa_086) | |
| I_170 | (S)—N-[1-(4-{2-Fluoro-4-[5-(isoxazol-3- | II_10 and 6- | rt: 6.721 |
| | ylaminomethyl)isoxazol-3-yl]phenyl}piperazin- | methylpyridine-3- | m/z: 562 |
| | 1-carbonyl)-2-methylpropyl]-6- | carboxylic acid | |
| | methylnicotinamide | (IIIa_086) | |
| I_171 | (R)—N-[2-(4-{2-Fluoro-4-[5-(isoxazol-3- | II_8 and IIIa_119 | rt: 6.025 |
| | ylaminomethyl)isoxazol-3-yl]phenyl}piperazin- | (3-aminopyridine-2- | m/z: 535 |
| | 1-yl)-1-methyl-2-oxoethyl]3-aminopyridine-2- | carboxylic acid) | |
| | carboxamide | | |
| I_172 | N-[2-(4-{2-Fluoro-4-[5-(isoxazol-3- | II_12 and IIIa_086 | rt: 4.643 |
| | ylaminomethyl)isoxazol-3-yl]phenyl}piperazin- | (6-methylpyridine- | m/z: 548 |
| | 1-yl)-1,1-dimethyl-2-oxoethyl]-6- | 3-carboxylic acid) | |
| | methylnicotinamide | | |
| I_173 | N-[1-(4-{2-Fluoro-4-[5-(isoxazol-3- | II_13 and IIIa_086 | rt: 5.017 |
| | ylaminomethyl)isoxazol-3-yl]phenyl}piperazine- | (6-methylpyridine- | m/z: 574 |
| | 1-carbonyl)cyclopentyl]-6-methylnicotinamide | 3-carboxylic acid) | |
| I_174 | 3-Amino-N-[1-(4-{2-fluoro-4-[5-(isoxazol-3- | II_13 and IIIa_120 | rt: 4.686 |
| | ylaminomethyl)isoxazol-3-yl]phenyl}piperazine- | (3-aminopyridine-4- | m/z: 575 |
| | 1-carbonyl)cyclopentyl]isonicotinamide | carboxylic acid) | |
| I_175 | (R)—N-[2-(4-{2-Fluoro-4-[5-(isoxazol-3- | II_8 and IIIa_067 | rt: 4.465 |
| | ylaminomethyl)isoxazol-3-yl]phenyl}piperazin- | (benzimidazole-5- | m/z: 559 |
| | 1-yl)-1-methyl-2-oxoethyl]-1H-benzoimidazole- | carboxylic acid) | |
| | 5-carboxamide | | |
| I_176 | (S)—N-[1-(4-{2-Fluoro-4-[5-(isoxazol-3- | II_10 and IIIa_067 | rt: 4.979 |
| | ylaminomethyl)isoxazol-3-yl]phenyl}piperazine- | (benzimidazole-5- | m/z: 587 |
| | 1-carbonyl)-2-methylpropyl]-1H- | carboxylic acid) | |
| | benzoimidazole-5-carboxamide | | |
| I_177 | (S)—N-[2-(4-{2-Fluoro-4-[5-(isoxazol-3- | II_7 and IIIa_055 | rt: 4.816 |
| | ylaminomethyl)isoxazol-3-yl]phenyl}piperazine- | (pyrazole-4- | m/z: 509 |
| | 1-yl)-1-methyl-2-oxoethyl]-1H-pyrazole-4- | carboxylic acid) | |
| | carboxamide | | |
| I_178 | (S)-3-Amino-N-[2-(4-{2-fluoro-4-[5-(isoxazol-3- | II_7 and IIIa_120 | rt: 4.431 |
| | ylaminomethyl)isoxazol-3-yl]phenyl}piperazin- | (3-aminopyridine-4- | m/z: 535 |
| | 1-yl)-1-methyl-2-oxoethyl]isonicotinamide | carboxylic acid) | |
| I_179 | (S)—N-[2-(4-{2-Fluoro-4-[5-(isoxazol-3- | II_7 and IIIa_119 | rt: 6.024 |
| | ylaminomethyl)isoxazol-3-yl]phenyl}piperazine- | (3-aminopyridine-2- | m/z: 535 |
| | 1-yl)-1-methyl-2-oxoethyl]-3-aminopyridine-2- | carboxylic acid) | |
| | carboxamide | | |
| I_180 | (S)—N-[2-(4-{2-Fluoro-4-[5-(isoxazol-3- | II_7 and IIIa_067 | rt: 4.470 |
| | ylaminomethyl)isoxazol-3-yl]phenyl}piperazine- | (benzimidazole-5 | m/z: 559 |
| | 1-yl)-1-methyl-2-oxoethyl]-1H-benzoimidazole- | carboxylic acid) | |
| | 5-carboxamide | | |
| I_181 | N-[2-(4-{2-Fluoro-4-[5-(isoxazol-3- | II_1 and IIIa_133 | rt: 4.179 |
| | ylaminomethyl)isoxazol-3-yl]phenyl}piperazin- | (imidazo[1,2- | m/z: 545 |
| | 1-yl)-2-oxoethyl]-imidazo[1,2-a]pyridine-6- | a]pyridine-6- | |
| | carboxamide | carboxylic acid) | |
| I_182 | N-[2-(4-{2-Fluoro-4-[5-(isoxazol-3- | II_1 and IIIa_134 | rt: 5.637 |
| | ylaminomethyl)isoxazol-3-yl]phenyl}piperazin- | (Thieno[2,3- | m/z: 562 |
| | 1-yl)-2-oxoethyl]thieno[2,3-b]pyridine-2- | b]pyridine-2- | |
| | carboxamide | carboxylic acid) | |
| I_183 | N-[2-(4-{2-Fluoro-4-[5-(isoxazol-3-ylamino- | II_1 and IIIa_135 | rt: 6.327 |
| | methyl)isoxazol-3-yl]phenyl}piperazin-1-yl)-2- | (7-nitroindole-2- | m/z: 589 |
| | oxoethyl]-7-nitro-1H-indole-2-carboxamide | carboxylic acid) | |
| I_184 | N-[2-(4-{2-Fluoro-4-[5-(isoxazol-3- | II_1 and IIIa_136 | rt: 6.318 |
| | ylaminomethyl)isoxazol-3-yl]phenyl}piperazin- | (5-nitrobenzofuran- | m/z: 590 |
| | 1-yl)-2-oxoethyl]-5-nitrobenzofuran-2- | 2-carboxylic acid) | |
| | carboxamide | | |
| I_185 | 6-Amino-N-(2-{4-[2-fluoro-4-(5- | II_11 and IIIa_083 | rt: 3.575 |
| | hydroxymethylisoxazol-3-yl)phenyl]piperazin-1- | (6-aminopyridine-3- | m/z: 455 |
| | yl}-2-oxoethyl)nicotinamide | carboxylic acid) | |
| I_186 | N-(2-{4-[2-Fluoro-4-(5-hydroxymethylisoxazol- | II_11 and IIIa_044 | rt: 5.444 |
| | 3-yl)phenyl]piperazin-1-yl}-2-oxoethyl)-5- | (5-methylthiophen- | m/z: 459 |
| | methylthiophene-2-carboxamide | 2-carboxylic acid) | |
| I_187 | N-(2-{4-[2-Fluoro-4-(5-hydroxymethylisoxazol- | II_11 and IIIa_050 | rt: 5.849 |
| | 3-yl)phenyl]piperazin-1-yl}-2- | (benzofuran-2- | m/z: 479 |
| | oxoethyl)benzofuran-2-carboxamide | carboxylic acid) | |
| I_188 | N-(2-{4-[2-Fluoro-4-(5-hydroxymethylisoxazol- | II_11 and IIIa_055 | rt: 3.991 |
| | 3-yl)phenyl]piperazin-1-yl}-2-oxoethyl)-1H- | (pyrazole-4- | m/z: 429 |
| | pyrazole-4-carboxamide | carboxylic acid) | |
| I_189 | N-(2-{4-[2-Fluoro-4-(5-hydroxymethylisoxazol- | II_11 and IIIa_066 | rt: 5.029 |
| | 3-yl)phenyl]piperazin-1-yl}-2-oxoethyl)-1H- | (indole-5-carboxylic | m/z: 478 |
| | indole-5-carboxamide | acid) | |
| I_190 | N-(2-{4-[2-Fluoro-4-(5-hydroxymethylisoxazol- | II_11 and IIIa_133 | rt: 3.643 |
| | 3-yl)phenyl]piperazin-1-yl}-2- | (imidazo[1,2- | m/z: 479 |
| | oxoethyl)imidazo[1,2-a]pyridine-6-carboxamide | a]pyridine-6- | |
| | | carboxylic acid) | |
| I_191 | N-(2-{4-[2-Fluoro-4-(5-hydroxymethylisoxazol- | II_11 and IIIa_086 | rt: 3.864 |
| | 3-yl)phenyl]piperazin-1-yl}-2-oxoethyl)-6- | (6-methylpyridine- | m/z: 454 |
| | methylnicotinamide | 3-carboxylic acid) | |
| I_192 | N-(2-{4-[2-Fluoro-4-(5-hydroxymethylisoxazol- | II_11 and IIIa_113 | rt: 5.752 |
| | 3-yl)phenyl]piperazin-1-yl}-2-oxoethyl)-8- | (8-hidroxyquinoline- | m/z: 506 |
| | hydroxyquinoline-2-carboxamide | 2-carboxylic acid) | |
| I_193 | N-(2-{4-[2-Fluoro-4-(5-hydroxymethylisoxazol- | II_11 and IIIa_007 | rt: 4.676 |
| | 3-yl)phenyl]piperazin-1-yl}-2-oxoethyl)-3- | (3-hidroxybenzoic | m/z: 455 |
| | hydroxybenzamide | acid) | |
| I_194 | 6-(Ethylmethylamino)-N-[2-(4-{2-fluoro-4-[5- | II_1 and IIIa_137 | rt: 4.478 |
| | (isoxazol-3-ylaminomethyl)isoxazol-3- | | m/z: 563 |
| | yl]phenyl}piperazin-1-yl)-2- | | |
| | oxoethyl]nicotinamide | | |
| I_195 | 6-Dimethylamino-N-[2-(4-{2-fluoro-4-[5- | II_1 and IIIa_138 | rt: 4.305 |
| | (isoxazol-3-ylaminomethyl)isoxazol-3- | | m/z: 549 |
| | yl]phenyl}piperazin-1-yl)-2- | | |
| | oxoethyl]nicotinamide | | |
| I_196 | N-[2-(4-{2-Fluoro-4-[5-(isoxazol-3- | II_1 and IIIa_139 | rt: 4.334 |
| | ylaminomethyl)isoxazol-3-yl]phenyl}piperazin- | | m/z: 579 |
| | 1-yl)-2-oxoethyl]-6-(2- | | |
| | methoxyethylamino)nicotinamide | | |
| I_197 | N-[2-(4-{2-Fluoro-4-[5-(isoxazol-3- | II_1 and IIIa_140 | rt: 4.150 |
| | ylaminomethyl)isoxazol-3-yl]phenyl}piperazin- | | m/z: 535 |
| | 1-yl)-2-oxoethyl]-6-methylaminonicotinamide | | |
| I_198 | N-[2-(4-{2-Fluoro-4-[5-(isoxazol-3- | II_1 and IIIa_141 | rt: 4.479 |
| | ylaminomethyl)isoxazol-3-yl]phenyl}piperazin- | | m/z: 536 |
| | 1-yl)-2-oxoethyl]-2-hydroxy-2-pyridin-3- | | |
| | ylacetamide | | |
| I_199 | 6-(Ethylmethylamino)-N-(2-{4-[2-fluoro-4-(5- | II_11 and IIIa_137 | rt: 4.002 |
| | hydroxymethylisoxazol-3-yl)phenyl]piperazin-1- | | m/z: 497 |
| | yl}-2-oxoethyl)nicotinamide | | |
| I_200 | N-(2-{4-[2-Fluoro-4-(5-hydroxymethylisoxazol- | II_11 and IIIa_139 | rt: 3.852 |
| | 3-yl)phenyl]piperazin-1-yl}-2-oxoethyl)-6-(2- | | m/z: 513 |
| | methoxyethylamino)nicotinamide | | |
| I_201 | N-(2-{4-[2-Fluoro-4-(5-hydroxymethylisoxazol- | II_11 and IIIa_142 | rt: 5.092 |
| | 3-yl)phenyl]piperazin-1-yl}-2-oxoethyl)-2- | (2-nitrobenzoic | m/z: 484 |
| | nitrobenzamide | acid) | |
| I_202 | 4-Amino-N-(2-{4-[2-fluoro-4-(5- | II_11 and IIIa_027 | rt: 4.128 |
| | hydroxymethylisoxazol-3-yl)phenyl]piperazin-1- | (4-amino-3- | m/z: 470 |
| | yl}-2-oxoethyl)-3-hydroxybenzamide | hidroxybenzoic | |
| | | acid) | |
| I_203 | 3-Cyano-N-(2-{4-[2-fluoro-4-(5- | II_11 and IIIa_143 | rt: 5.124 |
| | hydroxymethylisoxazol-3-yl)phenyl]piperazin-1- | (3-cyanobenzoic | m/z: 464 |
| | yl}-2-oxoethyl)benzamide | acid) | |
| I_204 | 3-Amino-N-(2-{4-[2-fluoro-4-(5- | II_11 and IIIa_120 | rt: 3.684 |
| | hydroxymethylisoxazol-3-yl)phenyl]piperazin-1- | (3-aminopyridine-4- | m/z: 455 |
| | yl}-2-oxoethyl)isonicotinamide | carboxylic acid) | |
| I_205 | N-(2-{4-[2-Fluoro-4-(5-hydroxymethylisoxazol- | II_11 and IIIa_110 | rt: 4.131 |
| | 3-yl)phenyl]piperazin-1-yl}-2- | (quinoline-5- | m/z: 490 |
| | oxoethyl)quinoline-5-carboxamide | carboxylic acid) | |
| I_206 | (S)-6-Amino-N-(2-{4-[2-fluoro-4-(5- | II_14 and IIIa_083 | rt: 3.800 |
| | hydroxymethylisoxazol-3-yl)phenyl]piperazin-1- | (6-aminopyridine-3- | m/z: 469 |
| | yl}-1-methyl-2-oxoethyl)nicotinamide | carboxylic acid) | |
| I_207 | (S)—N-(2-{4-[2-Fluoro-4-(5- | II_14 and IIIa_133 | rt: 3.856 |
| | hydroxymethylisoxazol-3-yl)phenyl]piperazin-1- | (imidazo[1,2- | m/z: 493 |
| | yl}-1-methyl-2-oxoethyl)imidazo[1,2-a]pyridine- | a]pyridine-6- | |
| | 6-carboxamide | carboxylic acid) | |
| I_208 | (S)—N-(2-{4-[2-Fluoro-4-(5- | II_14 and IIIa_135 | rt: 6.131 |
| | hydroxymethylisoxazol-3-yl)phenyl]piperazin-1- | (7-nitroindole-2- | m/z: 537 |
| | yl}-1-methyl-2-oxoethyl)-7-nitro-1H-indole-2- | carboxylic acid) | |
| | carboxamide | | |
| I_209 | (S)—N-[2-(4-{2-Fluoro-4-[5-(isoxazol-3- | II_7 and IIIa_133 | rt: 4.335 |
| | ylaminomethyl)isoxazol-3-yl]phenyl}piperazin- | (imidazo[1,2- | m/z: 559 |
| | 1-yl)-1-methyl-2-oxoethyl]imidazo[1,2- | a]pyridine-6- | |
| | a]pyridine-6-carboxamide | carboxylic acid) | |
| I_210 | (S)—N-[2-(4-{2-Fluoro-4-[5-(isoxazol-3- | II_7 and IIIa_135 | rt: 6.553 |
| | ylaminomethyl)isoxazol-3-yl]phenyl}piperazin- | (7-nitroindole-2- | m/z: 603 |
| | 1-yl)-1-methyl-2-oxoethyl]-7-nitro-1H-indole-2- | carboxylic acid) | |
| | carboxamide | | |
| I_211 | (S)—N-(2-{4-[2-Fluoro-4-(5- | II_14 and IIIa_045 | rt: 6.199 |
| | hydroxymethylisoxazol-3-yl)phenyl]piperazin-1- | (5-bromothiophen- | m/z: 538 |
| | yl}-1-methyl-2-oxoethyl)-5-bromothiophene-2- | 2-carboxylic acid) | |
| | carboxamide | | |
| I_212 | (S)—N-(2-{4-[2-Fluoro-4-(5- | II_14 and IIIa_076 | rt: 4.220 |
| | hydroxymethylisoxazol-3-yl)phenyl]piperazin-1- | (imidazo[1,2- | m/z: 493 |
| | yl}-1-methyl-2-oxoethyl)imidazo[1,2-a]pyridine- | a]pyridine-3- | |
| | 3-carboxamide | carboxylic acid) | |
| I_213 | (S)—N-(2-{4-[2-Fluoro-4-(5- | II_14 and IIIa_086 | rt: 4.112 |
| | hydroxymethylisoxazol-3-yl)phenyl]piperazin-1- | (6-methylpyridine- | m/z: 468 |
| | yl}-1-methyl-2-oxoethyl)-6-methylnicotinamide | 3-carboxylic acid | |

### Examples of Compounds of Formula Ia:

Compounds of formula Ia shown in table 5 were obtained by one of the methods 1-3 described below.

METHOD 1: To a 0.1 M solution of an amine of formula II (1 eq) in dried DMF an isocyanate of formula VIa (1.1 eq) was added. The reaction was stirred until the starting material disappeared on thin-layer chromatography. Water in an amount of about 10 parts by volume of DMF was added and the precipitate obtained was filtered and washed thoroughly with water. In case that no precipitate was formed, the mixture was extracted three times with EtOAc and then, the organic phases were washed twice with brine, dried over anhydrous sodium sulfate, filtered and concentrated at reduced pressure. If necessary, the obtained product was purified by column chromatography on silica gel.

METHOD 2: Corresponds to the method 2 described for the preparation of compounds of formula XI, using as starting materials an amine of formula II and an acyl chloride of formula VIb.

METHOD 3: To a 0.1 M solution of an amine of formula II (1 eq) in dried DMF, carbonyldiimidazole (1.1 eq) was added at room temperature. The reaction was stirred until the starting material disappeared on thin-layer chromatography. Then, an amine of formula VIc (1.5 eq) and triethylamine (1.5 eq) were added at room temperature. The reaction was stirred until the starting material disappeared on thin-layer chromatography. Water in an amount of about 10 parts by volume of DMF was added and the precipitate obtained was filtered and washed thoroughly with water. In case that no precipitate was formed, the mixture was extracted three times with EtOAc and then, the organic phases were washed twice with brine, dried over anhydrous sodium sulfate, filtered and concentrated at reduced pressure. If necessary, the obtained product was purified by column chromatography on silica gel.

TABLE 5

| Ex. | Name | Starting materials | HPLC-ESI-MS |
|---|---|---|---|
| Ia_1 | 1-[2-(4-{2-Fluoro-4-[5-(isoxazol-3- | II_1 and 4- | rt: 4.691 |
| | ylaminomethyl)isoxazol-3- | nitrophenyl | m/z: 565 |
| | yl]phenyl}piperazin-1-yl)-2-oxoethyl]-3-(4- | isocyanate (VIa_1) | |
| | nitrophenyl)urea | | |
| Ia_2 | 1-(3-Cyanophenyl)-3-[2-(4-{2-fluoro-4-[5- | II_1 and 3- | rt: 5.353 |
| | (isoxazol-3-ylaminomethyl)isoxazol-3- | cyanophenyl | m/z: 545 |
| | yl]phenyl}piperazin-1-yl)-2-oxoethyl]urea | isocyanate (VIa_2) | |
| Ia_3 | 1-[2-(4-{2-Fluoro-4-[5-(isoxazol-3- | II_1 and 3- | rt: 5.838 |
| | ylaminomethyl)isoxazol-3- | methoxyphenyl | m/z: 550 |
| | yl]phenyl}piperazin-1-yl)-2-oxoethyl]-3-(3- | isocyanate (VIa_3) | |
| | methoxyphenyl)urea | | |
| Ia_4 | 1-[2-(4-{2-Fluoro-4-[5-(isoxazol-3-ylamino- | II_1 and 3- | rt: 6.054 |
| | methyl)isoxazol-3-yl]phenyl}piperazin-1-yl)- | nitrophenyl | m/z: 565 |
| | 2-oxoethyl]-3-(3-nitrophenyl)urea | isocyanate (VIa_4) | |
| Ia_5 | 1-[2-(4-{2-Fluoro-4-[5-(isoxazol-3- | II_1 and 5-methyl- | rt: 6.580 |
| | ylaminomethyl)isoxazol-3- | 2-(trifluoromethyl)-3- | m/z: 592 |
| | yl]phenyl}piperazin-1-yl)-2-oxoethyl]-3-(5- | furyl isocyanate | |
| | methyl-2-trifluoromethylfuran-3-yl)urea | (VIa_5) | |
| Ia_6 | 1-(6-Fluoro-4H-benzo[1,3]dioxin-8-yl)-3-[2- | II_1 and 6-fluoro- | rt: 6.093 |
| | (4-{2-fluoro-4-[5-(isoxazol-3-ylaminomethyl)- | 4H-1,3-benzodioxin- | m/z: 596 |
| | isoxazol-3-yl]phenyl}piperazin-1-yl)-2- | 8-yl isocyanate | |
| | oxoethyl]urea | (VIa_6) | |
| Ia_7 | 1-Ethyl-3-[2-(4-{2-fluoro-4-[5-(isoxazol-3- | II_1 and ethyl | rt: 4.836 |
| | ylaminomethyl)isoxazol-3- | isocyanate (VIa_7) | m/z: 472 |
| | yl]phenyl}piperazin-1-yl)-2-oxoethyl]urea | | |
| Ia_8 | 3-[2-(4-{2-Fluoro-4-[5-(isoxazol-3- | II_1 and | rt: 4.787 |
| | ylaminomethyl)isoxazol-3- | dimethylcarbamoyl | m/z: 472 |
| | yl]phenyl}piperazin-1-yl)-2-oxoethyl]-1,1- | chloride (VIb_1) | |
| | dimethylurea | | |
| Ia_9 | 1-[2-(4-{2-Fluoro-4-[5-(isoxazol-3- | II_1 and isopropyl | rt: 5.157 |
| | ylaminomethyl)isoxazol-3- | isocyanate (VIa_8) | m/z: 486 |
| | yl]phenyl}piperazin-1-yl)-2-oxoethyl]-3- | | |
| | isopropylurea | | |
| Ia_10 | N-[2-(4-{2-fluoro-4-[5-(isoxazol-3-ylamino- | II_1 and 4- | rt: 4.753 |
| | methyl)isoxazol-3-yl]phenyl}piperazin-1-yl)- | morpholinocarbonyl | m/z: 514 |
| | 2-oxoethyl]morpholine-4-carboxamide | chloride (VIb_2) | |
| Ia_11 | [2-(4-{2-Fluoro-4-[5-(isoxazol-3- | II_1 and ammonium | rt: 4.369 |
| | ylaminomethyl)isoxazol-3- | chloride (VIc_1) | m/z: 444 |
| | yl]phenyl}piperazin-1-yl)-2-oxoethyl]urea | | |

### Examples of Compounds of Formula Ib:

Compounds of formula Ib shown in table 6 were obtained following the method 2 described for the preparation of compounds of formula XI, using an amine of formula IV and chloroformate of formula VIIa as starting materials.

TABLE 6

| Ex. | Name | Starting materials | HPLC-ESI-MS |
|---|---|---|---|
| Ib_1 | Ethyl N-[2-(4-{2-fluoro-4-[5-(isoxazol-3- | II_1 and ethyl | rt: 5.432 |
| | ylaminomethyl)isoxazol-3-yl]phenyl}- | chloroformate | m/z: 473 |
| | piperazin-1-yl)-2-oxoethyl]carbamate | (VIIa_1) | |
| Ib_2 | Vinyl N-[2-(4-{2-fluoro-4-[5-(isoxazol-3- | II_1 and vinyl | rt: 5.632 |
| | ylaminomethyl)isoxazol-3-yl]phenyl}- | chloroformate | m/z: 471 |
| | piperazin-1-yl)-2-oxoethyl]carbamate | (VIIa_2). | |
| Ib_3 | 4-Fluorophenyl N-[2-(4-{2-fluoro-4-[5- | II_1 and 4- | rt: 6.221 |
| | (isoxazol-3-ylaminomethyl)isoxazol-3- | fluorophenyl | m/z: 539 |
| | yl]phenyl}piperazin-1-yl)-2- | chloroformate (VIIa_3) | |
| | oxoethyl]carbamate | | |

### Tests of Antimicrobial Activity

In order to assess the antimicrobial activity of the compounds of the present invention a method of microdilution in microtiter plate was used. The compounds were diluted in a nutritious medium and, subsequently, distributed by two-fold serial dilutions in 96 well plates. Then, plates were inoculated with a bacterial suspension. After incubation for 24 h at 35° C. the minimum inhibitory concentration (MIC) of the drug in μg/mL was determined as the lowest concentration of compound which inhibits the growth of the bacterium. Results included in table 7 illustrate the antimicrobial activity of some of the compounds of the present invention in comparison with thus obtained with two compounds (linezolid and eperezolid) of a known antimicrobial activity. The antimicrobial activity of the compound versus Streptococcus faecalis (BCM-010, strain designation as for SALVAT collection) and Staphylococcus aureus (BCM-012, strain designation as for SALVAT collection), respectively, is shown in the different columns.

TABLE 7

| | BCM-010 | BCM-012 |
|---|---|---|
| COMPOUND | MIC (μg/mL) | MIC (μg/mL) |
| Linezolid | 4 | 2 |
| Eperezolid | 4 | 2 |
| I_1 | 2 | 1 |
| I_15 | 0.25-0.5 | 0.25-0.5 |
| I_32 | 0.25-0.5 | 0.25-0.5 |
| I_51 | 1 | 1 |
| I_58 | 0.5-1 | 0.25 |
| I_82 | 2 | 2 |
| I_84 | 2 | 1 |
| I_104 | 0.125-0.5 | 0.25-0.5 |
| I_117 | 1 | 1 |
| I_152 | 2 | 2 |
| I_153 | 2 | 2 |
| I_155 | 2 | 2 |
| I_160 | 0.25 | 0.25 |
| I_170 | 4 | 4 |
| I_171 | 1 | 2 |
| I_192 | 0.5 | 0.5 |
| I_213 | 1 | 1 |
| Ia_1 | 2 | 1 |

1. A compound of general formula I, 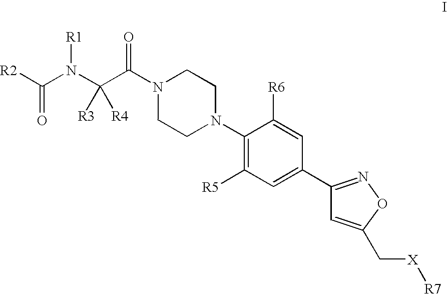
its stereoisomers and mixtures thereof, its polymorphs and mixtures thereof, N-oxides when there are oxidable nitrogen atoms, and the pharmaceutically acceptable solvates and addition salts of all of them, wherein:
X represents —O—, —NH—, —S—, —NHC(═O)— or —NHC(═S)—;
R1 represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra;
R2 represents —H, —ORb, —NRbRc, —(C₁-C₄)alkyl, —(C₂-C₄)alkenyl, —(C₂-C₄)alkynyl, or -Cy1 optionally substituted with one or more groups Rd or Re, wherein —(C₁-C₄)alkyl, —(C₂-C₄)alkenyl or —(C₂-C₄)alkynyl may be optionally substituted with one or more groups Rd and/or one group Rf;
R3 represents R1 or -Cy2 optionally substituted with one or more groups Ra or Rc;
R4 represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted by one or more halogen atoms;
alternatively, R3 and R4 may form together a 3- to 7-membered monocyclic ring, partially unsaturated, saturated or aromatic, containing from one to three heteroatoms independently selected from O, S and N, optionally substituted at any available position by one or more substituents Rc or halogen atoms;
R5 and R6 independently represent —H or halogen;
R7 represents R4 or heteroaryl optionally substituted with one or more groups Rc or halogen atoms, wherein heteroaryl represents a C- or N-radical of an aromatic 5- or 6-membered monocyclic ring containing from one to three heteroatoms independently selected from O, S and N;
each Ra independently represents halogen, ═O, —ORc, —OC(═O)Rc, ═CRcRc, —CN, —C(═O)Rc, —C(═O)ORc, —C(═O)NRcRc, —NO2, —NRcRc, —NRcC(═O)Rc, —NRcC(═O)ORc or —NRcC(═O)NRcRc;
Rb represents —H, Rg, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra and/or one group Rg;
each Rc independently represents —H, —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted by one or more halogen atoms;
each Rd independently represents halogen, ═CRaRc, ═CRcRc, —CN, —C(═O)Re′, —C(═O)ORe′, —C(═O)NRe′Rh′, —C(═O)SRe′, —C(═NRh′)NRe′Rh′, —C(═NRe′)NRh′Rh′, —C(═S)ORe′, —C(═O S)SRe′, —ORe′, ═O, —OC(═O)Re′, —OC(═O)NReRh′, —OC(═S)Re′, —O—N═O, —OSO₂Re, —NRe′Rh′, ═NRe′, ═N—CN, ═N—ORe′, —N⁺Re′Rh′Rh′, —N═NRe′, —NRh′-NRe′Re′, —NRe′-NRe′Rh′, —N₃, —N═O, —NRh′ORe′, —NRe′ORh′, —NO₂, —NRe′C(═O)Rh′, —NRh′C(═O)Re′, —NRh′C(═O)ORe, —NRe′C(═O)ORh, —NRh′C(═O)NReRh′, —NRe′C(═O)NRhRh′, —NRe′C(═O)NRh′NRh′Rh′, —NRh′C(═O)NReNRh′Rh′, —NRh′C(═O)NRh′NRe′Rh′, —NRe′SO₂Rh′, —NRh′SO₂Re′, —SRe′, —SORe′, —SO₂Re, —SO₂NRe′Rh′ or —SO₂ORe′;
each Re independently represents Rf or —(C₁-C₄)alkyl, —(C₂-C₄)alkenyl or —(C₂-C₄)alkynyl, wherein —(C₁-C₄)alkyl, —(C₂-C₄)alkenyl or —(C₂-C₄)alkynyl may be optionally substituted with one or more groups Ra and/or one group Rg;
each Re′ independently represents —H or —Re;
each Rf independently represents -Cy1 optionally substituted with one or more groups Ra or Rh;
each Rg independently represents -Cy1 optionally substituted with one or more groups Ra or Rc;
each Rh independently represents —(C₁-C₄)alkyl, —(C₂-C₄)alkenyl or —(C₂-C₄)alkynyl, all of them optionally substituted with one or more groups Ra;
each Rh′ independently represents —H or —Rh;
Cy1 represents a C- or N-radical of a 3- to 7-membered monocyclic or 6- to 10-membered bicyclic ring system, partially unsaturated, saturated or aromatic, containing from one to three heteroatoms independently selected from O, S and N; and
Cy2 represents a C- or N-radical of a 3- to 7-membered monocyclic ring, partially unsaturated, saturated or aromatic, containing from one to three heteroatoms independently selected from O, S and N.

2. The compound according to claim 1, wherein R1 represents —H or —(C₁-C₄)alkyl optionally substituted with one or more groups Ra.

3. The compound according to claim 2, wherein R1 represents —H.

4. The compound according to claim 1, wherein R2 represents —H, —(C₁-C₄)alkyl, —(C₂-C₄)alkenyl or —(C₂-C₄)alkynyl, wherein —(C₁-C₄)alkyl, (C₂-C₄)alkenyl or —(C₂-C₄)alkynyl may be optionally substituted with one or more groups Rd and/or one group Rf.

5. The compound according to claim 1, wherein R2 represents -Cy1 optionally substituted with one or more groups independently selected from —Re, halogen, ═CRaRc, ═CRcRc, —CN, —C(═O)Re′, —C(═O)ORe′, —C(═O)NRe′Rh′, ═O, —ORe′, —OC(═O)Re′, —NRe′Rh′, ═NRe′, —N⁺Re′Rh′Rh′, —N₃, —NRh′ORe′, —NRe′ORh′, —NO₂, —NRe′C(═O)Rh′, —NRh′C(═O)Re′, —NRe′C(═O)ORh′, —NRh′C(═O)ORe′, —NRe′C(═O)NRe′Rh′ or —NRh′C(═O)NRe′Rh′.

6. The compound according to claim 5, wherein Cy1 is selected from the group consisting of phenyl, a C- or N-radical of an aromatic 5- or 6-membered monocyclic ring containing from one to three heteroatoms independently selected from O, S and N, and a C- or N-radical of an aromatic bicyclic ring system containing from one to three heteroatoms independently selected from O, S and N, that comprises a 5- or 6-membered ring fused with a 5- or 6-membered ring, wherein all previously ring systems may be optionally substituted with —(C₁-C₄)alkyl, —(C₂-C₄)alkenyl, —(C₂-C₄)alkynyl, halogen, —CN, —C(═O)Re′, ═O, —ORe′, —NRe′Rh′, —NO₂, —NRe′C(═O)Rh′, —NRh′C(═O)Re′, wherein —(C₁-C₄)alkyl, —(C₂-C₄)alkenyl or —(C₂-C₄)alkynyl may be optionally substituted with one or more groups Ra.

7. The compound according to claim 1 to 6, wherein R3 represents —H or —(C₁-C₄)alkyl optionally substituted with one or more Ra and R4 represents —H or —(C₁-C₄)alkyl optionally substituted with one or more halogen atoms.

8. The compound according to claim 1, wherein R5 represents —F and R6 represents —H or —F.

9. The compound according to claim 1, wherein X represents —NH— and R7 represents heteroaryl optionally substituted with one or more groups Rc or halogen atoms, wherein heteroaryl represents a C- or N-radical of an aromatic 5- or 6-membered monocyclic ring containing from one to three heteroatoms independently selected from O, S and N.

10. A pharmaceutical composition comprising a therapeutically effective amount of a compound as defined in claim 1 and appropriate amounts of one or more pharmaceutically acceptable excipients.

11. A method of treating bacterial infections in an animal including a human comprising administering an effective amount of the compound as defined in claim 1.

12. The method according to claim 11, wherein the compound is administered topically or parenterally.
